SEQUENCE LISTING (28 sequences)

SEQ ID NO: 1 - DNA, 971 bp, Staphylococcus aureus
aactcggctcataaagatagtcaggatcaaaataaaaaagaacacgtggataaatcacaacagaaagataaacgcaatgtcaccaataaagataaaaatagcaccgcaccggatgacattggcaaaaacggtaaaatcaccaaacgtaccgaaacggtgtatgatgaaaaaacgaatattctgcagaacctgcaatttgatttcatcgatgacccgacctacgacaaaaatgtgctgctggttaaaaaacagggcagcattcattctaacctgaaattcgaaagtcacaaagaagagaaaaactccaactggctgaaatatccgtcagaataccatgtcgatttccaggtgaaacgtaatcgcaaaaccgaaattctggaccaactgccgaaaaacaaaatcagtaccgccaaagttgatagtacgttttcctatagctctggcggtaaattcgactctaccaaaggcatcggtcgtacgagttccaactcatactcgaaaaccatctcgtacaaccagcaaaactacgatacgatcgcaagcggcaaaaacaataactggcatgttcactggtctgtcattgctaacgatctgaaatatggcggtgaagttaaaaatcgcaacgacgaactgctgttttaccgtaatacccgcatcgcgacggtcgaaaacccggaactgtcattcgcgtcgaaatatcgttacccggccctggtgcgctccggttttaatccggaattcctgacctacctgagcaacgaaaaatctaacgaaaaaacgcagttcgaagtcacctatacgcgtaatcaagatattctgaaaaaccgtccgggcattcactacgcaccgccgatcctggagaaaaacaaagatggtcagcgcctgatcgtgacctatgaagttgactggaaaaacaaaaccgtgaaagtggtggacaaatactcggacgacaataaaccgtacaaagaagg

SEQ ID NO: 2 - protein, 323 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKE

SEQ ID NO: 3 - DNA, 927 bp, Staphylococcus aureus
aaaatcaacagcgaaatcaaacaagtcagcgaaaaaaatctggatggcgatacgaaaatgtacacgcgcacggcaaccacgagcgattcgcagaaaaacatcacccagagcctgcaatttaatttcctgaccgaaccgaactacgataaagaaacggtgttcatcaaagcaaaaggcaccatcggctcaggtctgcgtattctggacccgaatggctactggaactcgaccctgcgctggccgggtagctattctgtgagtattcagaatgttgatgacaacaataacaccaacgttacggattttgctccgaaaaatcaagatgaaagccgtgaagtcaaatatacctacggctataaaacgggcggtgatttctctatcaatcgcggcggtctgaccggtaatattacgaaagaatcgaactatagcgaaaccatctcctaccagcaaccgtcatatcgtaccctgctggatcagtccacgtcacataaaggcgttggttggaaagtcgaagcgcacctgatcaataacatgggccatgatcacacccgtcaactgacgaatgatagcgacaaccgcacgaaatctgaaatttttagtctgacccgcaatggtaacctgtgggcgaaagataacttcacgccgaaagacaaaatgccggtcaccgtgtccgaaggctttaatccggaattcctggccgttatgtctcatgataaaaaagacaaaggtaaaagtcagttcgtggttcactacaaacgttccatggatgaattcaaaatcgactggaaccgccatggcttctggggttactggagcggtgaaaaccacgtcgataaaaaagaagaaaaactgtctgcactgtatgaagtggactggaaaacccacaatgtcaaattcgtgaaagttctgaatgataatgaaaaaaaa

SEQ ID NO: 4 - protein, 309 aa, Staphylococcus aureus
KINSEIKQVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 5 - DNA, 966 bp, Staphylococcus aureus
gcaaacaaggactcccaggaccagaccaaaaaagaacacgtcgataaagcacagcagaaagaaaagcgtaatgtcaacgataaagataaaaataccccgggcccggatgacattggcaaaaacggcaaggttaccaaacgtaccgtcagtgaatatgacaaagaaaccaatattctgcagaacctgcaatttgatttcatcgatgacccgacgtacgacaaaaatgtgctgctggttaaaaagcaaggtagtatccattccaacctgaagtttgaaagccaccgtaatgaaaccaacgcgagttggctgaaatatccgtccgaataccatgtcgatttccaggtgcaacgcaatccgaaaacggaaattctggaccagctgccgaaaaacaagatctcaaccgcaaaagtggattcgacgtttagttattccctgggcggtaaattcgacagcaccaaaggcattggtcgcaccagcagcaacagctactcgaagagcatctcttacaaccagcaaaactacgataccatcgcaagcggcaaaaacaataaccgtcatgttcactggtctgtggttgctaatgatctgaagtatggtaacgaaatcaaaaatcgcaacgacgaatttctgttctaccgtaatacccgcctgagtacggtcgaaaacccggaactgtcatttgcgtcgaaatatcgttacccggccctggttcgctccggctttaatccggaatttctgacctacatcagcaacgaaaagtctaacgaaaagacgcgtttcgaagtgacctatacgcgcaatcaggatatcctgaaaaacaagccgggcattcactacggtcagccgatcctggaacaaaacaaagatggccagcgttttattgtcgtgtatgaagtggactggaaaaataagaccgttaaggttgtcgaaaaatattctgatcagaacaagccgtacaaagaaggt

SEQ ID NO: 6 - protein, 322 aa, Staphylococcus aureus
ANKDSQDQTKKEHVDKAQQKEKRNVNDKDKNTPGPDDIGKNGKVTKRTVSEYDKETNILQNLQFDFIDDPTYDKNVLLVKKQGSIHSNLKFESHRNETNASWLKYPSEYHVDFQVQRNPKTEILDQLPKNKISTAKVDSTFSYSLGGKFDSTKGIGRTSSNSYSKSISYNQQNYDTIASGKNNNRHVHWSVVANDLKYGNEIKNRNDEFLFYRNTRLSTVENPELSFASKYRYPALVRSGFNPEFLTYISNEKSNEKTRFEVTYTRNQDILKNKPGIHYGQPILEQNKDGQRFIVVYEVDWKNKTVKVVEKYSDQNKPYKEG

SEQ ID NO: 7 - DNA, 945 bp, Staphylococcus aureus
gcaagctcgtatgcggaaatcaaaagcaagatcaccaccgtctcagaaaagaacctggatggcgacaccaagatgtacacccgtaccgcgaccacgagcgatacggaaaagaaaattagccagtctctgcaatttaatttcctgaccgaaccgaactacgacaaagaaacggtgtttattaaagccaagggcaccatcggcagcggtctgaaaattctgaatccgaacggctactggaacagcaccctgcgttggccgggtagttattccgtttcaattcagaacgtcgatgacaacaataactcaaccaatgtcacggattttgcaccgaaaaaccaagacgaatcgcgtgaagtgaaatatacctacggctataagacgggcggtgatttcagtatcaatcgcggcggtctgaccggtaacatcacgaaggaaaagaactactcggaaaccatcagctaccagcaaccgtcttatcgtaccctgattgatcagccgaccacgaataaaggcgtcgcgtggaaggtggaagcccatagcatcaataacatgggtcatgatcacacccgtcaactgacgaacgactctgatgaccgcgtgaaatctgaaatttttagtctgacccgcaatggcaacctgtgggcaaaagataatttcacgccgaaaaacaagatgccggtgaccgtttccgaaggctttaatccggaatttctggctgttatgtcccatgataaaaacgacaaaggtaagtcacgtttcatcgtccactataaacgctcgatggatgactttaaactggattggaataagcatggcttctggggttactggagtggtgaaaaccacgttgaccagaaagaagaaaagctgtccgccctgtatgaagtggattggaaaacgcacgacgttaaactgattaagaccatcaacgataaagaacagaag

SEQ ID NO: 8 - protein, 315 aa, Staphylococcus aureus
ASSYAEIKSKITTVSEKNLDGDTKMYTRTATTSDTEKKISQSLQFNFLTEPNYDKETVFIKAKGTIGSGLKILNPNGYWNSTLRWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 9 - DNA, 954 bp, Staphylococcus aureus
gactcacaggaccaaaacaaaaaggaacacgttgataaggcacagcagaaagacaagcaagatagcaccaagaaaggcaaaaacgttgcggccccggatgacgtcggcaaaaacggcaaggtgaccaaacgtacggaaagcgaatacgatgaaaagaccaacatcctgcagaacctggaatttaatttcatcgatgacccgacctacgataaagacgtcctgctggtgaaaaagcaaggcagtattcattccaacctgaagttcgaaagtcacaaagaagaaaagaacagcacctggctgaaatatccgtcagaataccatgttgatttccaggtcaagcgtaacccgaaaaccgaaattctggaccaactgccgaaaaataagatcagtacggcaaaagtggattcaaccttttcgtatacgctgggcggtaaattcgactccattaaaggcatcggtcgcaatagctctaacagctattctcagaccatttcgtataatcagcaaaactacgatacgatcgcgagcggcaaaaacaataactggcatgtgcactggtctgttattgccaacgatctgaagtatggcggtgaagttaaaaatcgtaacgacgaatttctgttctaccgtaacacccgcacgagttccgttgataatccggaatcatcgtttgcagctaaatatcgttacccggcactggtccgcagtggttttaatccggaatttctgacctatctgagcaacgaaaagtctaatgaaaaaacgcagtttgaagtgacctatacgcgtaaccaagatatcctgaaaaatagcccgggcctgcattacgctccgccgattctggaaaagaacaaggttggtcaccgctttatcgtcacctatgaagtggattggaaaaataagacggtgaaggtggttgacaaatactctgatgaccagccgttccgcgaaggt

SEQ ID NO: 10 - protein, 318 aa, Staphylococcus aureus
DSQDQNKKEHVDKAQQKDKQDSTKKGKNVAAPDDVGKNGKVTKRTESEYDEKTNILQNLEFNFIDDPTYDKDVLLVKKQGSIHSNLKFESHKEEKNSTWLKYPSEYHVDFQVKRNPKTEILDQLPKNKISTAKVDSTFSYTLGGKFDSIKGIGRNSSNSYSQTISYNQQNYDTIASGKNNNWHVHWSVIANDLKYGGEVKNRNDEFLFYRNTRTSSVDNPESSFAAKYRYPALVRSGFNPEFLTYLSNEKSNEKTQFEVTYTRNQDILKNSPGLHYAPPILEKNKVGHRFIVTYEVDWKNKTVKVVDKYSDDQPFREG

SEQ ID NO: 11 - DNA, 927 bp, Staphylococcus aureus
aaaatcaaatcggaaatcacgcaagttagcgaacagaatatcgacggcaatacgaagatgtttacccgcacggcaacgacctcggatagccagaaaaagatcagccagtctctgcaatttaacttcctgaccgaaccgaactacgacaaggaaacggtgttcatcaaggcaaagggcaccatcggctctggtctgaaaattctggacccgaacggctactggaatagtaccctgcgttggccgggtagttattccgtgtcaatccagaacgttgataacaataccaatacgaaggttacggattttgccccgaaaaaccaagacgaaacccgcgaagtcaagtatacctacggctataaaacgggcggtgatttctcgattagcccgggcggtattaccggtaacatcacgaaagaacgtaattattctgaaaccatcagttaccagcaaccgagttatcgcaccctgattgaccagccggcgacgaataagggcgttggttggaaagtcgaagcccatctgatcaacaatatgggccatgatcacacccgtcaactgacgaacgattccgacaatcgcgtgggctcagaaatttttaccctgacgcgtaacggtaatctgtgggcgaaagataacttcacgccgaaaaataagatgccggtcaccgtgtccgaaggctttaacccggaatttctggccgttatgtcgcatgataaaaaggacaaaggcaagagcaaatttgtggttcactataaacgtacgatggatgactttaaaatcgattggatgcgccatggcttctggggttactggaccggtaaaaatcacgttgaccagaaggaagaaaaactgtctgcactgtatgaagtcgattggaaaacccacgacgtgaagttcattaaagctctggatgacaaagaaaagaaa

SEQ ID NO: 12 - protein, 309 aa, Staphylococcus aureus
KIKSEITQVSEQNIDGNTKMFTRTATTSDSQKKISQSLQFNFLTEPNYDKETVFIKAKGTIGSGLKILDPNGYWNSTLRWPGSYSVSIQNVDNNTNTKVTDFAPKNQDETREVKYTYGYKTGGDFSISPGGITGNITKERNYSETISYQQPSYRTLIDQPATNKGVGWKVEAHLINNMGHDHTRQLTNDSDNRVGSEIFTLTRNGNLWAKDNFTPKNKMPVTVSEGFNPEFLAVMSHDKKDKGKSKFVVHYKRTMDDFKIDWMRHGFWGYWTGKNHVDQKEEKLSALYEVDWKTHDVKFIKALDDKEKK

SEQ ID NO: 13 - protein, 315 aa, Staphylococcus aureus
ASSYAEIKSKITTVSEKNLDGDTKMYTRTATTSDTEKKISQSLQFNFLTEPNYDKETVFIKAKGTIGSGLKILNPNGYWNSTLTWPGSYSVSIQNVDDNNNSTNVTDFAPKNQDESREVKYTYGYKTGGDFSINRGGLTGNITKEKNYSETISYQQPSYRTLIDQPTTNKGVAWKVEAHSINNMGHDHTRQLTNDSDDRVKSEIFSLTRNGNLWAKDNFTPKNKMPVTVSEGFNPEFLAVMSHDKNDKGKSRFIVHYKRSMDDFKLDWNKHGFWGYWSGENHVDQKEEKLSALYEVDWKTHDVKLIKTINDKEQK

SEQ ID NO: 14 - protein, 314 aa, Staphylococcus aureus
ASSYAKINSEIKQVSEKNLDGETKMYTRTATTSDSQKNITQSLQFNFLTEPNYDKETVFIKAKGTIGSGLRILEPNGYWNSTLRWPGSYSVSIQNVDDNNNTNVTDFAPKNQDESREVKYTYGYKTGGDFSINRGGLTGNITKESNYSESISYQQPSYRTLLDQSTSNKGVGWKVEAHSINNMGHDHTRQLTNDSDDRVKSEIFSLTRNGNLWAKDNFTPKDKMPVTVSEGFNPEFLAVMSHDKKDEGKSKFVVHYKRSMDEFKIDWNKHGFWGYWSGENHVDKKEEKLSALYEVDWKTHNVRFIKVLNDKEQK

SEQ ID NO: 15 - protein, 307 aa, Staphylococcus aureus
NSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKNKMPVTVSEGFNPEFLAVMSHDKKDEGKSKFVVHYKRSMDEFKIDWNRHGFWGYWSGENHVDKKEEKLSALYEVDWKTHNVKFVKVLNDNEKK

SEQ ID NO: 16 - protein, 290 aa, Staphylococcus aureus
MYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDKGKSQFVVHYKRSMDEFKIDWNRHGFWGYWSGENHVDKKEEKLSALYEVDWKTHNVKFVKVLNDNEKK

SEQ ID NO: 17 - protein, 309 aa, Staphylococcus aureus
KINSEIKAVSEKNLDGDTKMYTRTATTSDSQKNITQSLQFNFITEPNYDKETVFIKAKGTIGSGLRILDPNGYWNSTLRWPGSYSVSIQNVDDNNHTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDEGKSKFVVHYKRSMDEFKIDWNRHGFWGYWSGENHVDKKEEKLSALYEVDWKTHDVKFVKVLNDNEKK

SEQ ID NO: 18 - protein, 309 aa, Staphylococcus aureus
KINSEIKQVSEKNLDGDTKMYTRTATTSDSQKNITQSLQFNFLTEPNYDKETVFIKAKGTIGSGLRILEPNGYWNSTLRWPGSYSVSIQNVDDNNNTNVTDFAPKNQDESREVKYTYGYKTGGDFSINRGGLTGNITKESNYSETISYQQPSYRTLLDQSTSNKGVGWKVEAHLINNMGHDHTRQLTNDSDNRTKSEIFSLTRNGNLWAKDNFTPKNKMPVTVSEGFNPEFLAVMSHDKKDKGKSKFVVHYKRSMDEFKIDWNRHGFWGYWSGENHVDKKEEKLSALYEVDWKTHDVKFVKVLNDNEKK

SEQ ID NO: 19 - protein, 309 aa, Staphylococcus aureus
KINSEIKQVSEKNLDGETKMYTRTATTSDSQKNITQSLQFNFLTEKNYDKETVFIKAKGTIGSGLRILEPNGYWNSTLRWPGSYSVSIQNVDDNNNTNVTDFAPKNQDESREVKYTYGYKTGGDFSINQGGLTGNITKESNYSETISYQQPSYRTLIDQPTTNKGVAWKVEAHLINNMGHDHTRQLTNDSDDRVKSEIFSLTRNGNLWAKDNFTPKNKMPVTVSEGFNPEFLAVMSHDKKDEGKSKFVVHYKRSMDEFKIDWNKHGFWGYWSGENHVDKKEEKLSALYEVDWKTHNVKFIKVLNDKEKK

SEQ ID NO: 20 - protein, 309 aa, Staphylococcus aureus
KINSEIKAVSEKNLDGDTKMYTRTATTSDSQKNITQSLQFNFLTEPNYDKETVFIKAKGTIGSGLRILDPNGYWNSTLRWPGSYSVSIQNVDDNNNTNVTDFAPKNQDESREVKYTYGYKTGGDFSINRGGLTGNITKESNYSETISYQQPSYRTLLDQSTSHKGVGWKVEAHLINNMGHDHTRQLTNDSDNRTKSEIFSLTRNGNLWAKDNFTPKDKMPVTVSEGFNPEFLAVMSHDKKDEGKSKFVVHYKRSMDEFKIDWNRHGFWGYWSGENHVDKKEEKLSALYEVDWKTHDVKFVKVLNDNEKK

SEQ ID NO: 21 - protein, 324 aa, Staphylococcus aureus
NSANKDSQDQTKKEHVDKAQQKEKRNVNDKDKNTPGPDDIGKNGKVTKRTETVYDEKTNILQNLQFDFIDDPTYDKNILLVKKQGSIHSNLKFESHKEENNSSWLKYPSEYHVDFQVKRNRKTEILDQLPKNKISTAKVDSTFSYNSGGKFDSVKGVGRTSSNSYSKTISYNQQNYDTIASGKNNNWHVHWSVVANDLKYGGEVKNRNDEFLFYRTTRLSTVENPELSFASKYRYPALVRSGFNPEFLTYLSNEKSNEKTQFEVTYTRNQDILKNKPGIHYAPPILEKNKDGQRLIVTYEVDWKNKTVKVIDKYSDDNKPYKEG

SEQ ID NO: 22 - protein, 324 aa, Staphylococcus aureus
NSANKDSQDQTKKEHVDKAQQKEKRNVNDKDKNTPGPDDIGKNGKVTKRTETVYDEKTNILQNLQFDFIDDPTYDKNVLLVKKQGSIHSNLKFESHKEENNSSWLKYPSEYHVDFQVKSNRKTEILDQLPKNKISTAKVDSTFSYNSGGKFDSVKGVGRTSSNSYSKTISYNQQNYDTIASGKNNNWHVHWSVVANDLKYGGEVKNRNDDFLFYRNTRLSTVENPELSFASKYRYPALVRSGFNPEFLTYLSNEKSNEKTQFEVTYTRNQDVLKNKPGIHYAPPILEKNKDGQRLIVTYEVDWKNKTVKVIDKYSDENKPYKEG

SEQ ID NO: 23 - protein, 324 aa, Staphylococcus aureus
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPSILEKNKDGQRLIVTYEVDWKNKTVKVVDKYSDDNKPYKEG

SEQ ID NO: 24 - protein, 324 aa, Staphylococcus aureus
NSAHKDSQDQNKKEHVDKSQQKDKRNVTNKDKNSTVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

SEQ ID NO: 25 - protein, 323 aa, Staphylococcus aureus
NSAHKDSQDQNKKEHVDKSQQKDKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYASPILEKNKEGQRLIVTYEVDWKNKTVKVVDKYSDNKSFREG

SEQ ID NO: 26 - protein, 294 aa, Staphylococcus aureus
NSAHKDSQDQNKKEHVDKSQQKDKRNVTNKDKNSPVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKIKMVKD

SEQ ID NO: 27 - protein, 323 aa, Staphylococcus aureus
NSAHKDSQDQNKKEHVDKSQQKEKRNVTNKDKNSTAPDDIGKNGKITKRTETVYDEKTNILQNLQFDFIDDPTYDKNVLLVKKQGSIHSNLKFESHKEEKNSNWLKYPSEYHVDFQVKRNPKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKEGQRLIVTYEVDWKNKTVKVVDKYSDNKSFREG

SEQ ID NO: 28 - protein, 324 aa, Staphylococcus aureus
NSAHKDSQDQNKKEHVDKSQQKEKRNVTNKDKNSPVPDDIGKNGKITKRTETVYDEKTNILQNLQFDFIDDPTYDKNVLLVKKQGSIHSNLKFESHKEEKNSNWLKYPSEYHVDFQVKRNRKTEILDQLPKNKISTAKVDSTFSYSSGGKFDSTKGIGRTSSNSYSKTISYNQQNYDTIASGKNNNWHVHWSVIANDLKYGGEVKNRNDELLFYRNTRIATVENPELSFASKYRYPALVRSGFNPEFLTYLSNEKSNEKTQFEVTYTRNQDILKNRPGIHYAPPILEKNKDGQRLIVTYEVDWKNKTVKVVDKYSDDNKPYKEG

(19) United States
(12) Patent Application Publication
(10) Pub. No.: US 2016/0108106 A1
(43) Pub. Date: Apr. 21, 2016

(54) GENERATION OF HIGHLY POTENT ANTIBODIES NEUTRALIZING THE LUKGH (LUKAB) TOXIN OF STAPHYLOCOCCUS AUREUS

(71) Applicant: ARSANIS BIOSCIENCES GMBH, Vienna (AT)

(72) Inventors: Eszter Nagy, Wien (AT); Adriana Badarau, Vienna (AT); Harald Rouha, Vienna (AT); Zoltán Magyarics, Vienna (AT); Sophia Zettl, Vienna (AT); Michael Benjamin Battles, Lebanon, NH (US)

(21) Appl. No.: 14/892,925

(22) PCT Filed: May 16, 2014
(86) PCT No.: PCT/EP2014/060123

§ 371 Date: Nov. 20, 2015

(30) Foreign Application Priority Data
May 21, 2013 (EP) 13168631.3

(51) Int. Cl. C07K 16/12; G01N 33/573; C07K 14/705

(52) U.S. Cl. CPC C07K 16/1271; C07K 14/70553; G01N 33/573; C07K 2317/32; C07K 2317/76

(57) ABSTRACT

The subject relates to an isolated Staphylococcus aureus leukocidin antigen comprising a LukGH complex, an antibody specifically binding to the Luk GH complex, and the human CD11b/CD18 complex for use in a method of determining the binding or toxicity of the Staphylococcus aureus Luk GH bi-component cytolysin.

### BACKGROUND OF THE INVENTION

Staphylococcus aureus (S. aureus) is one of most important human pathogens that can cause a broad spectrum of infections in humans ranging from asymptomatic colonization and mild skin infections to severe deep tissue infections, pneumonia, blood stream infections and sepsis. This pathogen uses multiple virulence mechanisms to cause disease and interfere with host defense. One of its most potent virulence factors are the leukocidins specialized in killing white blood cells, especially phagocytic cells. LukGH (also called LukAB) is the most recently identified leukocidin that is able to lyse polymorphonuclear cells (PMNs), monocytes and dendritic cells (Dumont et al, 2011; Ventura et al, 2010) and also to activate them to produce pro-inflammatory cytokines. US20110274693A1 describes Luk A or LukB antibodies, and in particular anti-LukA polyclonal antibodies.

LukGH is a bi-component cytolysin, similarly to HIgAB, HIgCB, LukED and LukSF (PVL). LukH (S-component) and LukG (F-component) display approximately 30 and 40% amino acid homology with the S and F components of the above mentioned bi-component leukocidins, respectively.

Malachova et al. (The Journal of Infectious Diseases, Epub 2012 Aug. 7, vol. 206, no. 8, pp 1185-1193) describe purification of LukGH, which is obtained as two separate bands by SDS-PAGE analysis.

LukGH is the most variable bi-component S. aureus toxin. While LukSF, LukED and HIgABC are highly conserved in different S. aureus strains, LukGH exhibits up to 14% amino acid changes. This level of amino acid differences almost reaches the one observed between two different toxins, eg HIgC vs LukS or LukS vs HIgAC or LukE (˜16% difference). There are very few data about the function of LukGH and those are generated with two sequences from the Newman and LAC (USA300) strains that are almost identical to each other. It is not known whether the other variants are active with human cells or not, especially two sequences derived from the two S. aureus genomes, MRSA252 (EMRSA16) and MSHR1132 (“silver” S. aureus) that are considered to be the most different from USA300 and other S. aureus clonal complexes.

Based on the PMN lysis activity of S. aureus cultures supernatants, LukGH seems to be one of the most potent leukotoxin for human innate cells (Dumont et al, 2013, Malachowa et al, 2012). Therefore, it is plausible that inhibiting LukGH's toxin function by neutralizing antibodies has a positive effect during S. aureus disease and supports host defense by saving the phagocytic cells migrating to the site of infections. It is our aim to develop human therapeutics to prevent and treat human S. aureus infections with monoclonal antibodies neutralizing the LukGH toxins.

Based on literature, it is the S component of bi-component leukocidins that recognizes a cell surface receptor, and this interaction induces a conformational change leading to the binding of the F-component and formation of the octameric membrane spanning pore structure described for LukSF and HIgAB (Colin, Infect Immun, 1994:3184; Meunier, Biochim Biophys Acta, 1997:275).

### SUMMARY OF THE INVENTION

It is the objective of the present invention to provide for an antibody directed against the S. aureus cytotoxin LukGH (also called LukAB) that is cross-reactive among the different variants of this toxin and provides cross neutralizing potency.

The object is solved by the subject of the present invention.

According to the invention there is provided an isolated Staphylococcus aureus leukocidin antigen comprising a LukGH complex.

Specifically, the LukGH complex comprises the LukG and LukH components as a dimer or oligomer. Specifically, the LukGH complex is a heterodimeric or oligomeric LukGH antigen. Such antigen is specifically provided as a heterodimer or oligomer which is soluble in the aqueous phase, in particularly which is not bound to the surface of a cell that is susceptible to cell lysis upon LukGH binding, such as PMNs, monocytes or dendritic cells.

Specifically, the LukGH complex is composed of recombinant proteins and/or proteins derived from S. aureus strains.

Specifically, the LukG and LukH components are co-expressed by a recombinant host cell, purified from native sources and/or co-refolded.

Specifically, the antigen is provided as a protein complex in the soluble form.

Specifically, the antigen is capable of binding to the human CD11b/CD18 receptor.

According to the invention, there is further provided an antibody specifically binding to the LukGH complex. It specifically can be demonstrated that binding of the antibody of the invention to the heterodimeric or oligomeric LukGH antigen was far improved as compared to binding of the separated (monomeric) LukG or LukH.

Specifically, the antibody is capable of neutralizing the LukGH complex.

Specifically, the antibody is binding to the LukGH complex derived from the USA300 clone, preferably from the TCH1516 strain, and at least one of the LukGH complex variants.

Specifically, the LukGH complex variants have at least one point mutation in the amino acid sequences of any of the LukG or LukH components, as compared to the LukGH complex derived from the USA300 clone, e.g. a change in one or more of the amino acid residues in the sequence. Even the very different LukGH complex variants derived from MRSA252 and MSHR1132 strains may be cross-specifically bound by the antibody of the invention, and cross-neutralized.

Specifically, the LukGH complex derived from a USA300 clone comprises the LukG component comprising the amino acid sequence of SEQ ID 4 and/or the LukH component comprising the amino acid sequence of SEQ ID 2.

Specifically, the LukGH complex variants comprise a LukG component comprising an amino acid sequence selected from the group consisting of SEQ ID 8, 12, 13, 14, 15, 16, 17, 18, 19 and 20, and/or a LukH component comprising an amino acid sequence selected from the group consisting of SEQ ID 6, 10, 21, 22, 23, 24, 25, 26, 27 and 28.

Specifically, the antibody is cross-neutralizing the LukGH complex and the LukGH complex variants.

According to the invention, there is further provided the human CD11b/CD18 complex for use in a method of determining the binding or toxicity of the Staphylococcus aureus LukGH bi-component cytolysin.

Specifically, the CD11b/CD18 complex is used in its native form, or isolated or immobilized forms.

According to the invention there is specifically provided a cross-neutralizing antibody comprising at least one polyspecific binding site that binds to LukGH from USA300 clone (eg strain TCH_1516) and at least one of the LukGH variants. Specifically the LukGH toxin is selected from the group consisting of genes expressed by the EMRSA16 MRSA252 strain or the MSHR1132 strain.

Unexpectedly, applying the same approach that has worked to generate neutralizing antibodies for other bi-component leukotoxins of S. aureus was found to be ineffective for the LukGH toxin. The different mode of action of this toxin is herein described and the toxin complex is specifically provided for selection and generation of antibodies, instead of the single, non-toxic components.

Specifically, the antibody of the invention is a full-length monoclonal antibody or an antibody fragment thereof comprising at least one antibody domain incorporating the binding site, preferably an antibody selected from the group consisting of murine, chimeric, humanized or human antibodies, heavy-chain antibodies, Fab, Fd, scFv and single-domain antibodies like VH, VHH or VL, preferably a human IgG1 antibody.

The antibody preferably has an affinity to bind the LukGH complex with a Kd of less than 10⁻⁸M, preferably less than 10⁻⁹M.

According to a specific aspect, the antibody exhibits in vitro neutralization potency in a cell-based assay with an IC50 of less than 50:1 mAb:toxin ratio (mol/mol), preferably less than 10:1, more preferably less than 1:1.

According to a further specific aspect, the antibody neutralizes the targeted toxins in animals, including both, human and non-human animals, and inhibits S. aureus pathogenesis in vivo, preferably any models of pneumonia, bacteremia or sepsis, peritonitis and osteomyelitis.

According to a further aspect, the invention provides for a method of producing an antibody of the invention, wherein a recombinant host cell of the invention is cultivated or maintained under conditions to produce said antibody.

According to a further aspect, the invention provides for a method of identifying a candidate protective antibody comprising:

(a) providing a sample containing an antibody or antibody-producing cell; and

(b) assessing for binding of an antibody in or produced by the sample with the LukGH complex of the invention, wherein a positive reaction between the antibody and the LukGH complex identifies the antibody as candidate protective antibody.

According to a further aspect, the invention provides for a method of producing an antibody of the invention, comprising

(a) providing a candidate protective antibody identified according to the identification method of the invention; and

(b) producing a monoclonal antibody, or a humanized or human form of the candidate protective antibody, or a derivative thereof with the same epitope binding specificity as the candidate protective antibody.

According to a further aspect, the invention provides for a method of producing an antibody of the invention, comprising

(a) immunizing a non-human animal with the LukGH complex of the invention;

(b) forming immortalized cell lines from the isolated B-cells;

(c) screening the cell lines obtained in b) to identify a cell line producing a monoclonal antibody that binds to the LukGH complex; and

(d) producing the monoclonal antibody, or a humanized or human form of the antibody, or a derivative thereof with the same epitope binding specificity as the monoclonal antibody.

According to a further aspect, the invention provides for a method of producing an antibody of the invention, comprising

(a) immunizing a non-human animal with the LukGH complex of the invention;

(b) forming immortalized cell lines from the isolated B-cells;

(c) screening the cell lines to identify a cell line producing a monoclonal antibody that binds to the LukGH complex and at least one of the LukGH complex variants; and

(d) producing the monoclonal antibody, or a humanized or human form of the antibody, or a derivative thereof with the same epitope binding specificity as the monoclonal antibody.

According to a further aspect, the invention provides for an antibody of the invention for medical use, including human medical and veterinary use. Specifically the antibody is provided for use in treating a subject at risk of or suffering from a S. aureus infection comprising administering to the subject an effective amount of the antibody to limit the infection in the subject, to ameliorate a disease condition resulting from said infection or to inhibit S. aureus pneumonia pathogenesis.

Specifically the antibody is provided for protecting against S. aureus infections.

According to a specific aspect, there is further provided a method of treatment wherein a subject at risk of or suffering from a S. aureus infection is treated, which method comprises administering to the subject an effective amount of the antibody to limit the infection in the subject, to ameliorate a disease condition resulting from said infection or to inhibit S. aureus pneumonia pathogenesis.

Specifically, the method of treatment is provided for protecting against pathogenic S. aureus.

According to a specific embodiment, the antibody is administered in a parenteral or mucosal formulation.

According to a further aspect, the invention provides for a pharmaceutical preparation of an antibody of the invention, preferably comprising a parenteral or mucosal formulation, optionally containing a pharmaceutically acceptable carrier or excipient.

According to a further aspect, the invention provides for an antibody of the invention, for diagnostic use to detect any S. aureus infections, including high toxin producing MRSA infections, such as necrotizing pneumonia, and toxin production in furunculosis and carbunculosis.

Specifically, the antibody is provided for use according to the invention, wherein a systemic infection with S. aureus in a subject is determined ex vivo by contacting a sample of body fluid of said subject with the antibody, wherein a specific immune reaction of the antibody determines the infection.

According to a specific aspect, there is further provided a method of diagnosing an S. aureus infection in a subject, including high toxin producing MRSA infections, such as necrotizing pneumonia, and toxin production in furunculosis and carbunculosis.

Specifically, the method of diagnosing is provided, wherein a systemic infection with S. aureus in a subject is determined ex vivo by contacting a sample of body fluid of said subject with the antibody, wherein a specific immune reaction of the antibody determines the infection.

According to a further aspect, the invention provides for a diagnostic preparation of an antibody of the invention, optionally containing the antibody with a label and/or a further diagnostic reagent with a label.

According to a further aspect, the invention provides for an immunogen comprising:
(a) the LukGH complex of the invention;
(b) optionally further epitopes or antigens not natively associated with said LukGH complex of (a); and
(c) a carrier.

Specifically, the carrier is a pharmaceutically acceptable carrier, preferably comprising buffer and/or adjuvant substances.

The immunogen of the invention is preferably provided in a vaccine formulation, preferably for parenteral use.

Specifically the immunogen of the invention is provided for medical use, specifically for use in treating a subject by administering an effective amount of said immunogen to protect the subject from an S. aureus infection, to prevent a disease condition resulting from said infection or to inhibit S. aureus pneumonia pathogenesis.

Specifically the immunogen of the invention is provided for eliciting a protective immune response.

According to a specific aspect, there is further provided a method of treatment wherein a subject at risk of a S. aureus infection is treated, which method comprises administering to the subject an effective amount of the immunogen to prevent infection in the subject, in particular to protect against pathogenic S. aureus.

According to a further aspect, the invention provides for an isolated nucleic acid encoding an antibody of the invention, or encoding an antigen of the invention, in particular encoding the LukGH complex or fusion protein.

### FIGURES

FIG. 1: A: Determining LukGH toxicity with human phagocytic cells, as described in example 3, using the three most divergent LukG and LukH sequence types derived from the USA300_TCH1516, MRSA252 (CC30, ST36) and MSHR1132 (CC75, ST1850) strains. B: Relatedness of LukG and LukH sequences from different S. aureus genomes; C: Amino acid sequence alignment of LukG and LukH sequence variants (sequences see FIG. 12):

LukG: ST36 MRSA252—SEQ ID 8;

ST30_E1410—SEQ ID 13;

CC133_ED133—SEQ ID 4;

ST59_M013—SEQ ID 20;

ST1_MW2—SEQ ID 15;

ST8_COL—SEQ ID 16;

ST5_USA300_TCH1516—SEQ ID 4;

ST239_JKD6159—SEQ ID 18;

ST398_0385—SEQ ID 14;

CC10/ST10_H19—SEQ ID 19;

ST1850_MSHR1132—SEQ ID 12.

LukH: ST5_Mu50-o—SEQ ID 23;

ST5_N315—SEQ ID 24;

ST8_USA300TCH1516—SEQ ID 2;

ST239_JKD6159—SEQ ID 28;

ST80_11819-97—SEQ ID 25;

ST22_EMRSA-15—SEQ ID 27;

ST59_M013—SEQ ID 26;

ST1850_MSHR1132—SEQ ID 10;

ST10_H19—SEQ ID 21;

ST398_ST398—SEQ ID 22;

ST36_MRSA252—SEQ ID 6.

FIG. 2: Lack of neutralization of the LukGH toxin by antibodies generated with LukG or LukH, as described in example 3.

FIG. 3: Detecting neutralization of the LukGH toxin by antibodies generated with LukG or LukH if mAbs are first bound to single components, as described in example 3.

FIG. 4: LukH and LukG forms a complex without being in contact with target cells, as described in example 4.

FIG. 5: LukH and LukG exist as dimer in solution, as described in example 4.

FIG. 6: Highly potent LukGH neutralizing antibodies discovered by selection with the LukGH complex using differentiated HL-60 cells, as described in example 5. A: tested with recombinant LukGH_TCH1516 complex; B: tested with native LukGH secreted by S. aureus USA300_TCH1516 and present in bacterial culture supernatant.

FIG. 7: Highly potent LukGH neutralizing antibodies discovered by selection with the LukGH complex using freshly isolated human PMNs, as described in example 5. A: tested with recombinant LukGH_TCH1516 complex; B: tested with recombinant LukGH_MRSA252 complex. C. Binding of the LukGH antibodies to LukG, LukH and LukGH variants in Forte-Bio as described in example 5.

FIG. 8: LukGH toxin neutralizing mAbs inhibit binding of LukGH to human phagocytic cells, as described in example 5.

FIG. 9: The CD11 b/CD18 complex is identified as the human LukGH receptor, as described in example 6. A: Silverstained SDS-PAGE gel with purified proteins; B: Mass spectromeztry analysis of tryptic peptides; C: Western blot analysis of purified complexes using anti-human CD11 b antibody.

FIG. 10: A. Structure of the LukGH octamer with LukG as black and LukH as grey cartoon. B. LukG-LukH interface 1 with the residues involved in electrostatic interactions shown as sticks and salt bridges as dotted lines. C. LukG-LukH interface 2 with the residues involved in electrostatic interactions (labelled in the inset) shown as sticks and salt bridges as dotted lines.

FIG. 11: A. Binding of LukG to biotinylated LukH or LukH2 immobilized on streptavidin sensors in Forte-Bio. B. Cross-linking of LukG and LukH (WT and variants, 35 μg/ml each) in 20 mM Hepes pH 7.5 plus 50 mM NaCl in presence of 1 mM glutaraldehyde after incubation at 37° C. for the duration indicated monitored by SDS-PAGE.

FIG. 12: Sequence IDs

SEQ ID 1: LukH nucleotide sequence of the USA300 TCH1516 strain

SEQ ID 2: LukH amino acid sequence of the USA300 TCH1516 strain

SEQ ID 3 LukG nucleotide sequence of the USA300 TCH1516 strain

SEQ ID 4: LukG amino acid sequence of the USA300 TCH1516 strain

SEQ ID 5 LukH nucleotide sequence of the MRSA252 strain (Genbank, accession number BX571856)

SEQ ID 6: LukH amino acid sequence of the MRSA252 strain

SEQ ID 7 LukG nucleotide sequence of the MRSA252 strain

SEQ ID 8: LukG amino acid sequence of the MRSA252 strain

SEQ ID 9 LukH nucleotide sequence of the MSHR1132 strain (Genbank, accession number FR821777)

SEQ ID 10: LukH amino acid sequence of the MSHR1132 strain

SEQ ID 11 LukG nucleotide sequence of the MSHR1132 strain

SEQ ID 12: LukG amino acid sequence of the MSHR1132 strain

SEQ ID 13: LukG ST30_E1410 amino acid sequence

SEQ ID 14: LukG ST398_0385 amino acid sequence

SEQ ID 15: LukG ST1_MW2 amino acid sequence

SEQ ID 16: LukG ST8_COL amino acid sequence

SEQ ID 17: LukG CC133_ED133 amino acid sequence

SEQ ID 18: LukG ST239_JKD6159 amino acid sequence

SEQ ID 19: LukG CC10/ST10_H19 amino acid sequence

SEQ ID 20: LukG ST59_M013 amino acid sequence

SEQ ID 21: LukH ST10_H19 LukH amino acid sequence

SEQ ID 22: LukH ST398_5 T398 amino acid sequence

SEQ ID 23: LukH ST5_Mu50-o amino acid sequence

SEQ ID 24: LukH ST5_N315 amino acid sequence

SEQ ID 25: LukH ST80_11819-97 amino acid sequence

SEQ ID 26: LukH ST59_M013 amino acid sequence

SEQ ID 27: LukH ST22_EMRSA-15 amino acid sequence

SEQ ID 28: LukH ST239_JKD6159 amino acid sequence

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VL/VH pair, an antibody comprising a VLNH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VLNH pair, an antibody comprising or consisting of a VL/VH domain pair and constant antibody domains, such as heavy-chain antibodies, Fab, F(ab′), (Fab)₂, scFv, Fd, Fv, or a full-length antibody, e.g. of an IgG type (e.g., an IgG1, IgG2, IgG3, or IgG4 subtype), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” further applies to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. The human antibody of the invention may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals, e.g. mammalians including human, that comprises genes or sequences from different origin, e.g. chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/or a fusion protein, in which any domain of the antibody may be fused at any position of one or more other proteins, such as other antibodies, e.g. a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g. PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

The preferred derivatives as described herein are functionally active with regard to the antigen binding, preferably which have a potency to neutralize S. aureus and/or which are protective antibodies.

It is understood that the term “antibody” also refers to variants of an antibody.

The term “variant” shall particularly refer to antibodies, such as mutant antibodies or fragments of antibodies, e.g. obtained by mutagenesis methods, in particular to delete, exchange, introduce inserts into a specific antibody amino acid sequence or region or chemically derivatize an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomisation techniques. In some cases positions are chosen randomly, e.g. with either any of the possible amino acids or a selection of preferred amino acids to randomise the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of an antibody as used herein, means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence, and which modification does not affect (in particular impair) the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. Specifically the functionally active variants of an antibody of the invention has the polyspecific binding site that binds to Hla and at least one of the bi-component toxins of S. aureus, as further described herein.

Functionally active variants may be obtained, e.g. by changing the sequence of a parent antibody, but with modifications within an antibody region besides the binding site, or derived from a parent antibody, by a modification that does not impair the antigen binding, and preferably would have a biological activity similar to the parent antibody, including the ability to bind the LukGH complex of S. aureus and/or to neutralize S. aureus with a specific potency, e.g. with substantially the same biological activity, as determined by a specific binding assay or functional test to target S. aureus or S. aureus toxins. The term “substantially the same biological activity” as used herein refers to the activity as indicated by substantially the same activity being at least 50%, at least 60%, at least 70%, at least 80%, at least 90%, at least 95%, at least 98% or even at least 100% or at least 110%, or at least 120%, or at least 130%, or at least 140%, or at least 150%, or at least 160%, or at least 170%, or at least 180%, or at least 190%, e.g. up to 200% of the activity as determined for the parent antibody.

In a preferred embodiment the functionally active variant of a parent antibody

a) is a biologically active fragment of the antibody, the fragment comprising at least 50% of the sequence of the molecule, preferably at least 70%, more preferably at least 80%, still more preferably at least 90%, even more preferably at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally occurring allelic variants, as well as mutants or any other non-naturally occurring variants. As is known in the art, an allelic variant is an alternate form of a (poly) peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties. The preferred variants as described herein are functionally active with regard to the antigen binding, preferably which have a potency to neutralize S. aureus and/or which are protective antibodies.

An antibody of the present invention may or may not exhibit Fc effector function. Though the mode of action is mainly mediated by neutralizing antibodies without Fc effector functions, Fc can recruit complement and aid elimination of the target antigen, such as a toxin, from the circulation via formation of immune complexes.

Specific antibodies may be devoid of an active Fc moiety, thus, either composed of antibody domains that do not contain an Fc part of an antibody or that do not contain an Fcgamma receptor binding site, or comprising antibody domains lacking Fc effector function, e.g. by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis, e.g. mutations in the Fcgamma receptor binding site or by derivatives or agents to interfere with ADCC and/or CDC activity of an antibody format, so to achieve reduction or increase of Fc effector function.

A significant reduction of Fc effector function is typically understood to refer to Fc effector function of less than 10% of the unmodified (wild-type) format, preferably less than 5%, as measured by ADCC and/or CDC activity.

A significant increase of Fc effector function is typically understood to refer to an increase in Fc effector function of at least 10% of the unmodified (wild-type) format, preferably at least 20%, 30%, 40% or 50%, as measured by ADCC and/or CDC activity.

The term “glycoengineered” variants with respect to antibody sequences shall refer to glycosylation variants having modified immunogenic properties, ADCC and/or CDC as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC). Removal of N-Glycan at N297, eg through mutating N297, e.g. to A, or T299 typically results in aglycosylated antibody formats with reduced ADCC.

Major differences in antibody glycosylation occur between cell lines, and even minor differences are seen for a given cell line grown under different culture conditions. Expression in bacterial cells typically provides for an aglycosylated antibody. CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180). In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are interposed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping. Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen.

Herein the term “epitope” shall particularly refer to the single epitope recognized by an antibody, or the mixture of epitopes comprising epitope variants, each recognized by a cross-reactive antibody.

Specifically, the epitope recognized by an antibody targeting the LukGH complex is located within the antigenic region formed by the binding of LukG to LukH, e.g. located on such protein domains that are in contact with both LukH and LukG, e.g. the rim or the cap domain, but still accessible, thus, an epitope of the LukGH complex particularly recognized by an antibody of the invention is formed by the LukG and LukH components when complexed to form a dimer or oligomer in solution, and exposed or accessible for being bound by an antibody.

Specifically, the epitope is located on such protein domains that are otherwise in contact with the receptor of a susceptible cell, and inhibiting the binding of the soluble LUKGH complex to its putative cellular receptor upon antibody binding to the soluble LukGH complex. Thus, the antibody directed against such epitope of a LukGH complex would bind to the LukGH complex in competition to the receptor binding.

Specifically, the epitope is located on such protein domains that are accessible on the soluble LukGH complex only, thus, not accessible when LukGH is bound to a susceptible cell.

The term “expression” is understood in the following way. Nucleic acid molecules containing a desired coding sequence of an expression product such as e.g. an antibody as described herein, and control sequences such as e.g. a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions, e.g. for the expression of a protein coded for by foreign DNA carried by the vector and introduced to the host cell.

Coding DNA is a DNA sequence that encodes a particular amino acid sequence for a particular polypeptide or protein such as e.g. an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “LukGH complex” as used herein shall refer to the dimer or oligomer, including 1:1 dimers, or any other ratio of the LukG and the LukH components, preferably a complex comprising at least 1 LukG and at least 1 LukH component, or at least 2, or at least 3, or at least 4 of each or any of the LukG or LukH components or of both LukG and LukH components. The LukGH dimer is understood as a LukGH complex comprising 1 LukG and 1 LukH molecule bound to form a dimeric complex. The LukGH tetramer is understood as a LukGH complex comprising 2 LukG and 2 LukH molecules bound to form a tetrameric complex. The LukGH hexamer is understood as a LukGH complex comprising 3 LukG and 3 LukH molecules bound to form a hexameric complex. The LukGH octamer is understood as a LukGH complex comprising 4 LukG and 4 LukH molecules bound to form an octameric complex. The trimeric or pentameric LukGH complex typically comprises a ratio different from 1:1, e.g. 1 LukG and 2 LukH molecules, or vice versa, to form a trimer, or 2 LukG and 3 LukH molecules, or vice versa, to form a pentamer.

An “immune response” to a composition, e.g. an immunogenic composition, herein also termed “immunogen” comprising an antigen or epitope, or a vaccine as described herein is the development in the host or subject of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

A “protective immune response” is understood as an immune response that provides a significantly better outcome of an induced or natural infection or toxin challenge in comparison to that of the non-immune population. Protective immune response against toxins is mainly mediated by neutralizing antibodies having high affinity, e.g. with a Kd of less than 10⁻⁸M. The benefit of neutralization of toxins is the protection of targets cells and prevention of inflammation. Fc mediated immune complex formation can contribute as well by removing the toxin from the circulation (via the RES cells).

An immunogen or immunogenic composition usually comprises the antigen or epitope and a carrier, which may specifically comprise an adjuvant. The term “adjuvant” refers to a compound that when administered in conjunction with an antigen augments and/or redirects the immune response to the antigen, but when administered alone does not generate an immune response to the antigen. Adjuvants can augment an immune response by several mechanisms including lymphocyte recruitment, stimulation of B and/or T cells, and stimulation of macrophages. Exemplary carriers are liposomes or cationic peptides; exemplary adjuvants are aluminium phosphate or aluminium hydroxide, MF59 or CpG oligonucleotide.

The term “isolated” or “isolation” as used herein with respect to a nucleic acid, an antibody or other compound shall refer to such compound that has been sufficiently separated from the environment with which it would naturally be associated, so as to exist in “substantially pure” form. “Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those chemically synthesized.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as antibodies or epitopes of the invention, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated—for example, the polypeptides or polynucleotides can be mixed with pharmaceutically acceptable carriers or excipients when used in diagnosis or therapy.

In particular, the isolated LukGH complex of the invention is isolated from a physiological surface, e.g. a cellular surface, where the LukG and LukH would be immobilized to form a pore-forming LukGH bicomponent toxin on the surface of a cell susceptible to cell lysis by such LukGH bicomponent toxin.

The term “neutralizing” or “neutralization” is used herein in the broadest sense and refers to any molecule that inhibits a pathogen, such as S. aureus from infecting a subject, or to inhibit the pathogen from promoting infections by producing potent protein toxins, or to inhibit the toxins from damaging a target cell in a subject, irrespective of the mechanism by which neutralization is achieved. Neutralization can be achieved, e.g., by an antibody that inhibits the binding and/or interaction of the S. aureus toxin(s) with its cognate receptor on target cells. In certain embodiments, the antibodies described herein can neutralize the toxin activity wherein the in vivo or in vitro effects of the interaction between the toxin and the target cell, such as red blood cells are reduced or eliminated. Neutralization can further occur by inhibition of forming active toxin, for example in the case of the S. aureus bi-component cytolysins, by inhibition of binding of the S- and F-components or formation of the oligomeric pores in cytomembranes.

The neutralization potency of antibodies against cytolytic toxins is typically determined in a standard assay by measuring increased viability or functionality of cells susceptible to the given toxin. Neutralization can be expressed by percent of viable cells with and without antibodies. For highly potent antibodies, a preferred way of expressing neutralization potency is the antibody/toxin molar ratio, where lower values correspond to higher potency. Values below 1 define very high potency.

The term “cross-neutralizing” as used herein shall refer to neutralizing the major variants of the LukGH complex, including the LukGH complex of the USA300 clone and at least one of the LukGH variants.

The term “Staphylococcus aureus” or “S. aureus” or “pathogenic S. aureus” is understood in the following way. Staphylococcus aureus bacteria are normally found on the skin or in the nose of people and animals. The bacteria are generally harmless, unless they enter the body through a cut or other wound. Typically, infections are minor skin problems in healthy people. Historically, infections were treated by broad-spectrum antibiotics, such as methicillin. Now, though, certain strains have emerged that are resistant to methicillin and other beta-lactam antibiotics, such as penicillin and cephalosporins. They are referred to as methicillin-resistant Staphylococcus aureus (also known as multi-drug resistant Staphylococcus aureus, or “MRSA”).

S. aureus infections, including MRSA, generally start as small red bumps that resemble pimples, boils or spider bites. These bumps or blemishes can quickly turn into deep, painful abscesses that require surgical draining. Sometimes the bacteria remain confined to the skin. On occasion, they can burrow deep into the body, causing potentially life-threatening infections in a broad range of human tissue, including skin, soft tissue, bones, joints, surgical wounds, the bloodstream, heart valves, lungs, or other organs. Thus, S. aureus infections can result in disease conditions associated therewith, which are potentially fatal diseases, such as necrotizing fasciitis, endocarditis, sepsis, toxic shock syndrome, and various forms of pneumonia, including necrotizing pneumonia, and toxin production in furunculosis and carbunculosis. MRSA infection is especially troublesome in hospital or nursing home settings where patients are at risk of or prone to open wounds, invasive devices, and weakened immune systems and, thus, are at greater risk for infection than the general public.

Antibodies neutralizing S. aureus toxins are interfering with the pathogens and pathogenic reactions, thus able to limit or prevent infection and/or to amleiorate a disease condition resulting from such infection, or to inhibit S. aureus pathogenesis, in particular pneumonia pathogenesis. In this regard “protective antibodies” are understood herein as neutralizing antibodies that are responsible for immunity to an infectious agent observed in active or passive immunity. In particular, protective antibodies as described herein are able to neutralize toxic effects (such as cytolysis, induction of pro-inflamamtory cytokine expresseion by target cells) of secreted virulence factors (exotoxins) and hence interfere with pathogenic potential of S. aureus.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation.

As used herein, the term “specificity” or “specific binding” refers to a binding reaction which is determinative of the cognate ligand of interest in a heterogeneous population of molecules. Thus, under designated conditions (e.g. immunoassay conditions), an antibody specifically binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different, preferably the difference is at least 100 fold, and more preferred a least 1000 fold.

The term is also applicable where e.g. an antibody is specific for a particular epitope which is cross-reactive a number of antigens, in which case the specific antibody will be able to bind to the various antigens carrying the cross-reactive epitope. Such binding site of an antibody or and antibody with a specificity to bind a cross-reactive epitope is also called a polyspecific or cross-specific binding site and antibody, respectively. For example, an antibody may have a polyspecific binding site specifically binding an epitope cross-reactive a number of different antigens with sequence homology within the epitope and/or structural similarities to provide for a conformational epitope of essentially the same structure, e.g. cross-reactive at least the Hla and a bi-component toxin of S. aureus.

The imrnunospecificity of an antibody, its binding capacity and the attendant affinity the antibody exhibits for a cross-reactive binding sequence, are determined by a cross-reactive binding sequence with which the antibody immunoreacts (binds). The cross-reactive binding sequence specificity can be defined, at least in part, by the amino acid residues of the variable region of the heavy chain of the immunoglobulin the antibody and/or by the light chain variable region amino acid residue sequence.

Use of the term “having the same specificity”, “having the same binding site” or “binding the same epitope” indicates that equivalent monoclonal antibodies exhibit the same or essentially the same, i.e. similar immunoreaction (binding) characteristics and compete for binding to a pre-selected target binding sequence. The relative specificity of an antibody molecule for a particular target can be relatively determined by competition assays, e.g. as described in Harlow, et al., ANTIBODIES: A LABORATORY MANUAL, Cold Spring Harbor Laboratory Press, Cold Spring Harbor, N. Y., 1988).

The term “subject” as used herein shall refer to a warm-blooded mammalian, particularly a human being or a non-human animal. MRSA is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a S. aureus infection. The subject may be a patient at risk of a S. aureus infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g. treated for prophylaxis or therapy of S. aureus disease conditions. In particular, the subject is treated, which is either at risk of infection or developing such disease or disease reccurrence, or a subject that is suffering form such infection and/or disease associated with such infection.

Specifically the term “prophylaxis” refers to preventive measures which is intended to encompass prevention of the onset of pathogenesis or prophylactic measures to reduce the risk of pathogenesis.

Specifically, the method for treating, preventing, or delaying a disease condition in a subject as described herein, is by interfering with the pathogenesis of S. aureus as causal agent of the condition, wherein the pathogenesis includes a step of forming a pore on the subject's cellular membrane, e.g. by the specific virulence factors or toxins.

The virulence of S. aureus is due to a combination of numerous virulence factors, which include surface-associated proteins that allow the bacterium to adhere to eukaryotic cell membranes, a capsular polysaccharide that protects it from opsonophagocytosis, and several exotoxins. S. aureus causes disease mainly through the production of secreted virulence factors such as hemolysins, enterotoxins and toxic shock syndrome toxin. These secreted virulence factors suppress the immune response by inactivating many immunological mechanisms in the host, and cause tissue destruction and help establish the infection. The latter is accomplished by a group of pore forming toxins, the most prominent of which is Hla, a key virulence factor for S. aureus pneumonia.

S. aureus produces a diverse array of further virulence factors and toxins that enable this bacterium to neutralize and withstand attack by different kinds of immune cells, specifically subpopulations of white blood cells that make up the body's primary defense system. The production of these virulence factors and toxins allow S. aureus to maintain an infectious state. Among these virulence factors, S. aureus produces several bi-component leukotoxins, which damage membranes of host defense cells and erythrocytes by the synergistic action of two non-associated proteins or subunits. Among these bi-component toxins, gamma-hemolysin (HIgAB and HIgCB) and the Pantone-Valentine Leukocidin (PVL) are the best characterized.

The toxicity of the leukocidins towards mammalian cells involves the action of two components. The first subunit is named class S component, and the second subunit is named class F component. The S and F subunits act synergistically to form pores on white blood cells including monocytes, macrophages, dendritic cells and neutrophils (collectively known as phagocytes). The repertoire of bi-component leukotoxins produced by S. aureus is known to include cognate and non-cognate pairs of the F and S components, e.g. LukGH.

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like).

The term “therapeutically effective amount”, used herein interchangeably with any of the terms “effective amount” or “sufficient amount” of a compound, e.g. an antibody or immunogen of the present invention, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of S. aureus or S. aureus pathogenesis.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

The antibody or the immunogen of the present invention may be used prophylactically to inhibit onset of S. aureus infection, or therapeutically to treat S. aureus infection, particularly S. aureus infections such as MRSA that are known to be refractory or in the case of the specific subject, have proven refractory to treatment with other conventional antibiotic therapy.

A therapeutically effective amount of the antibody as described herein, such as provided to a human patient in need thereof, may specifically be in the range of 0.5-500 mg, preferably 1-400 mg, even more preferred up to 300 mg, up to 200 mg, up to 100 mg or up to 10 mg, though higher doses may be indicated e.g. for treating acute disease conditions.

Moreover, a treatment or prevention regime of a subject with a therapeutically effective amount of the antibody of the present invention may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

An effective amount of an immunogen as described herein, such as provided to a patient at risk of developing a disease condition associated with an S. aureus infection, may specifically be in the range of 1-15 mg/kg per dose.

For example the immunogen may be administered as a first dose followed by one or more booster dose(s), within a certain timeframe, according to a prime-boost immunization scheme to induce a long-lasting, efficacious immune response to S. aureus infection. A preferred vaccination schedule would encompass administration of three doses, e.g. a first dose on day 0, a second dose on day 5-40, and a third dose on day 10-100, preferably on days 0, 28 and 90. According to a preferred accelerated schedule the administration may be on days 0, 7 and 14. Accelerated schedules may be indicated for prophylaxis, e.g. for patients facing elective surgery. Usually alum is used as an adjuvant, e.g. as phosphate or hydroxide.

Preferred antibodies of the invention are specifically binding said LukGH complex antigens with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or K_{D}). Usually a binder is considered a high affinity binder with a Kd<10⁻⁸ M, preferably a Kd<10⁻⁹ M, even more preferred is a Kd<10⁻¹⁰ M.

Yet, in a particularly preferred embodiment the antigen binding affinities are of medium affinity, e.g. with a Kd of less than 10⁻⁶ and up to 10⁻⁸ M, e.g. when binding to at least two antigens.

Medium affinity binders may be provided according to the invention, preferably in conjunction with an affinity maturation process, if necessary.

Affinity maturation is the process by which antibodies with increased affinity for a target antigen are produced. Any one or more methods of preparing and/or using affinity maturation libraries avaiable in the art may be employed in order to generate affinity matured antobodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody according to the invention exhibits at least a 10 fold increase in affinity of binding, preferably at least a 100 fold increase. The affinity maturation may be employed in the course of the selection campaigns employing respective libraries of parent molecules, either with antibodies having medium binding affinity to obtain the antibody of the invention having the specific target binding property of a binding affinity Kd<10⁻⁸ M. Alternatively, the affinity may be even more increased by affinity maturation of the antibody according to the invention to obtain the high values corresponding to a Kd of less than 10⁻⁹ M, preferably less than 10⁻¹⁰ M or even less than 10⁻¹¹ M, most preferred in the picomolar range.

Phagocytic effector cells may be activated through another route employing activation of complement. Antibodies that bind to surface antigens on microorganisms attract the first component of the complement cascade with their Fc region and initiate activation of the “classical” complement system. This results in the stimulation of phagocytic effector cells, which ultimately kill the target by complement dependent cytotoxicity (CDC).

According to a specific embodiment, the antibody of the invention has a cytotoxic activity in the presence of immune-effector cells as measured in a standard ADCC or CDC assay. A cytotoxic activity as determined by either of an ADCC or CDC assay may be shown for an antibody of the invention, if there is a significant increase in the percentage of cytolysis as compared to a control. The cytotoxic activity related to ADCC or CDC is preferably measured as the absolute percentage increase, which is preferably higher than 5%, more preferably higher than 10%, even more preferred higher than 20%.

The LukGH complex of the invention may be used for selecting antibodies from an antibody library, e.g. a yeast-displayed antibody library.

Screening methods for identifying antibodies with the desired neutralizing properties may be inhibition of toxin binding to the target cells, inhibition of formation of dimers or oligomers. Inhibition of pore formation, inhibition of cell lysis, inhibition of the induction of cytokines, lymphokines, and any pro-inflammatory signaling, and/or inhibition of in vivo effect on animals (death, hemolysis, overshooting inflammation, organ dysfunction. Reactivity can be assessed based on direct binding to the desired toxins, or differential binding to the LukGH complex as compared to binding of the separate LukG or LukH (monomeric) antigens, e.g. using standard assays.

Once anti-LukGH antibodies with the desired properties have been identified, the dominant epitope or epitopes recognized by the antibodies may be determined. Methods for epitope mapping are well-known in the art and are disclosed, for example, in Epitope Mapping: A Practical Approach, Westwood and Hay, eds., Oxford University Press, 2001.

Epitope mapping concerns the identification of the epitope to which an antibody binds. There are many methods known to those of skill in the art for determining the location of epitopes on proteins, including crystallography analysis of the antibody-antigen complex, competition assays, gene fragment expression assays, and synthetic peptide-based assays. An antibody that “binds the same epitope” as a reference antibody is herein understood in the following way. When two antibodies recognize epitopes that are identical or sterically overlapping epitopes, the antibodies are referred to as binding the same or essentially the same or substantially the same epitopes. A commonly used method for determining whether two antibodies bind to identical or sterically overlapping epitopes is the competition assay, which can be configured in all number of different formats, using either labeled antigen or labeled antibody. Usually, an antigen is immobilized on a 96-well plate, and the ability of unlabeled antibodies to block the binding of labeled antibodies is measured using radioactive or enzyme labels.

Once antibodies with the desired neutralizing properties are identified, such antibodies, including antibody fragments can be produced by methods well-known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and transfecting a recombinant host cell with the coding sequences for expression, using well known recombinant expression vectors, e.g. the plasmids of the invention or expression cassette(s) comprising the nucleotide sequences encoding the antibody sequences. Recombinant host cells can be prokaryotic and eukaryotic cells, such as those described above.

According to a specific aspect, the nucleotide sequence may be used for genetic manipulation to humanize the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the target toxins and greater efficacy against S. aureus. It will be apparent to one of skill in the art that one or more polynucleotide changes can be made to the antibody and still maintain its binding ability to the target toxins.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101:189-198) describe the production of monoclonal antibodies from separately produced heavy and light chains, using plasmids expressed in separate cultures of E. coli. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

In another aspect, the invention provides an isolated nucleic acid comprising a sequence that codes for production of the recombinant antibody of the present invention.

In another aspect, the invention provides an isolated nucleic acid comprising a sequence that codes for production of the recombinant epitope of the present invention, or a molecule comprising such epitope of the present invention. However, the epitope of the invention may also be synthetically produced, e.g. through any of the synthesis methods well-known in the art.

An antibody or epitope encoding nucleic acid can have any suitable characteristics and comprise any suitable features or combinations thereof. Thus, for example, an antibody or epitope encoding nucleic acid may be in the form of DNA, RNA, or a hybrid thereof, and may include nonnaturally-occurring bases, a modified backbone, e.g., a phosphothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

The invention moreover provides pharmaceutical compositions which comprise an antibody or an immunogen as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tabletted or encapsulated for conventional administration. Alternatively, an antibody may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody or immunogen of the present invention and one or more therapeutically active agents are formulated. Stable formulations of the antibody or immunogen of the present invention are prepared for storage by mixing said immunoglobulin having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody or immunogen of the present invention, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, e.g. employing inhalable technology or pulmonary delivery systems, vaginally, parenterally, rectally, or intraocularly.

Exemplary formulations as used for parenteral administration include those suitable for subcutaneous, intramuscular or intravenous injection as, for example, a sterile solution, emulsion or suspension.

In one embodiment, the antibody or immunogen of the present invention is the only therapeutically active agent administered to a subject, e.g. as a disease modifying or preventing monotherapy.

Alternatively, the antibody or immunogen of the present invention is administered in combination with one or more other therapeutic or prophylactic agents, including but not limited to standard treatment, e.g. antibiotics, steroid and non-steroid inhibitors of inflammation, and/or other antibody based therapy, e.g. employing antibacterial or anti-inflammatory agents.

A combination therapy is particularly employing a standard regimen, e.g. as used for treating MRSA infection. This may include antibiotics, e.g. tygecycline, linezolide, methicillin and/or vancomycin.

In a combination therapy, the antibody may be administered as a mixture, or concomitantly with one or more other therapeutic regimens, e.g. either before, simultaneously or after concomitant therapy.

Prophylactic administration of immunogens in some cases may employ a vaccine comprising the immunogen of the present invention, i.e. a monovalent vaccine. Yet, a multivalent vaccine comprising different immunogens to induce an immune response against the same or different target pathogens may be used.

The biological properties of the antibody, the immunogen or the respective pharmaceutical preparations of the invention may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibody, immunogen and respective pharmaceutical componsitions of the present invention may be tested in humans to determine their therapeutic or prophylactic efficacy, toxicity, immunogenicity, pharmacokinetics, and/or other clinical properties.

The invention also provides the subject antibody of the invention for dagnostic purposes, e.g. for use in methods of detecting and quantitatively determining the concentration of a toxin or antibody as immunoreagent or target in a biological fluid sample.

The invention also provides methods for detecting the level of toxins or S. aureus infection in a biological sample, such as a body fluid, comprising the step of contacting the sample with an antibody of the invention. The antibody of the invention may be employed in any known assay method, such as competitive binding assays, direct and indirect sandwich assays, immunoprecipitation assays and enzyme-linked immunosorbent assays (ELISA).

A body fluid as used according to the present invention includes biological samples of a subject, such as tissue extract, urine, blood, serum, stool and phlegm.

In one embodiment the method comprises contacting a solid support with an excess of a certain type of antibody fragment which specifically forms a complex with a target, such as at least one of the toxins targeted by the antibody of the invention, conditions permitting the antibody to attach to the surface of the solid support. The resulting solid support to which the antibody is attached is then contacted with a biological fluid sample so that the target in the biological fluid binds to the antibody and forms a target-antibody complex. The complex can be labeled with a detectable marker. Alternatively, either the target or the antibody can be labeled before the formation the complex. For example, a detectable marker (label) can be conjugated to the antibody. The complex then can be detected and quantitatively determined thereby detecting and quantitatively determining the concentration of the target in the biological fluid sample.

For particular applications the antibody of the invention is conjugated to a label or reporter molecule, selected from the group consisting of organic molecules, enzyme labels, radioactive labels, colored labels, fluorescent labels, chromogenic labels, luminescent labels, haptens, digoxigenin, biotin, metal complexes, metals, colloidal gold and mixtures thereof. Antibodies conjugated to labels or reporter molecules may be used, for instance, in assay systems or diagnostic methods, e.g. to diagnose S. aureus infection or disease conditions associated therewith.

The antibody of the invention may be conjugated to other molecules which allow the simple detection of said conjugate in, for instance, binding assays (e.g. ELISA) and binding studies.

Another aspect of the present invention provides a kit comprising an antibody, which may include, in addition to one or more antibodies, various diagnostic or therapeutic agents. A kit may also include instructions for use in a diagnostic or therapeutic method. Such instructions can be, for example, provided on a device included in the kit, e.g. tools or a device to prepare a biological sample for diagnostic purposes, such as separating a cell and/or protein containing fraction before determining the respective toxin(s) to diagnose a disease. Advantageously, such a kit includes an antibody and a diagnostic agent or reagent that can be used in one or more of the various diagnostic methods described herein. In another preferred embodiment, the kit includes an antibody, e.g. in the lyophilized form, in combination with pharmaceutically acceptable carrier(s) that can be mixed before use to form an injectable composition for near term administration.

### EXAMPLES

### Example 1

### Generation of Recombinant Toxins

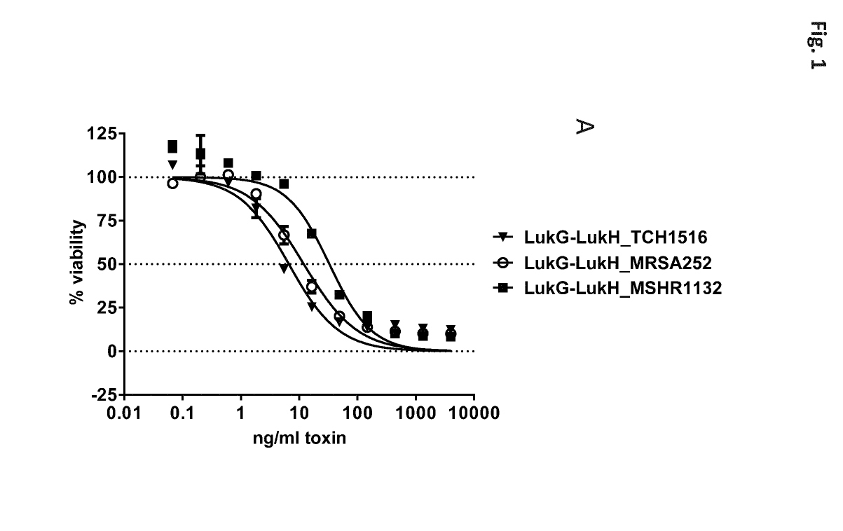

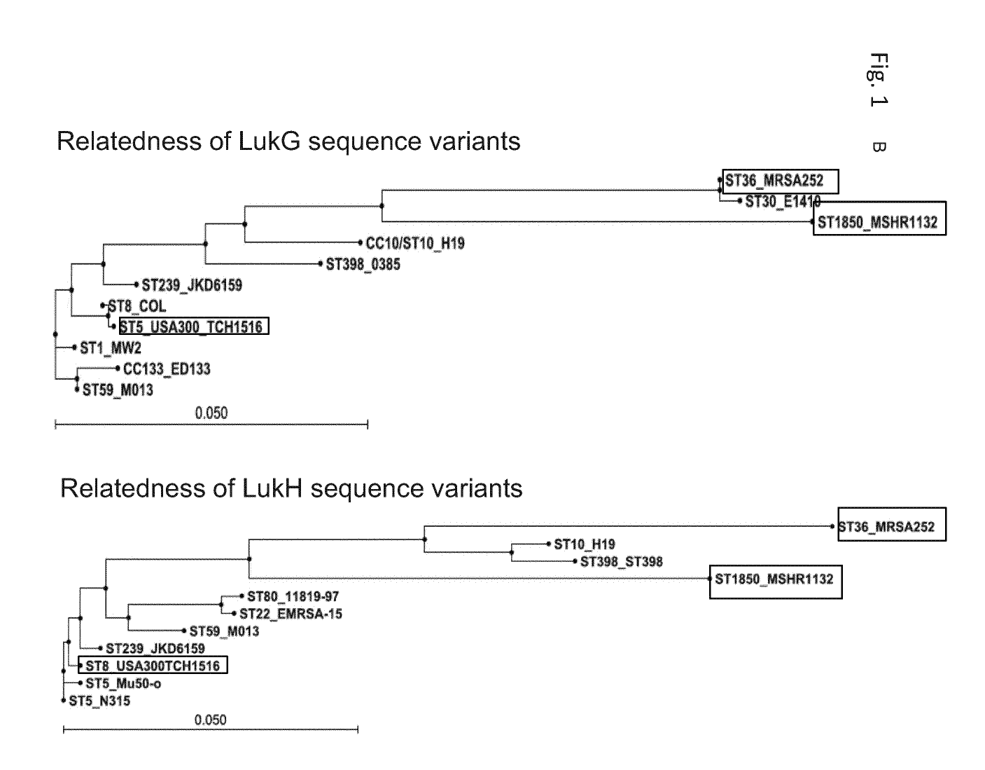

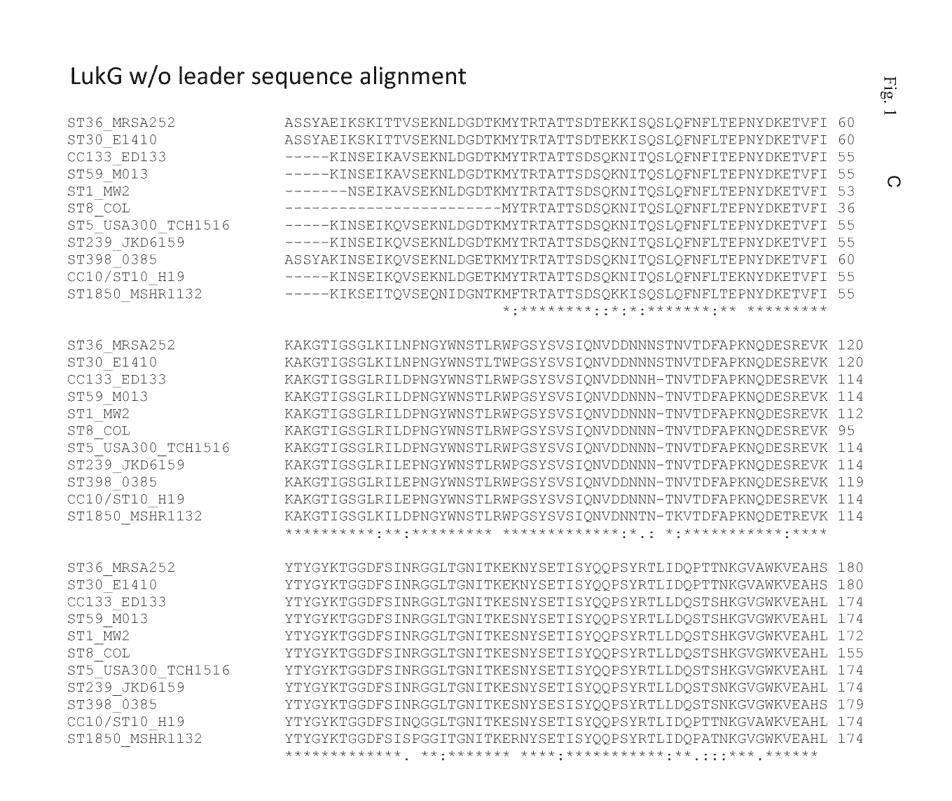

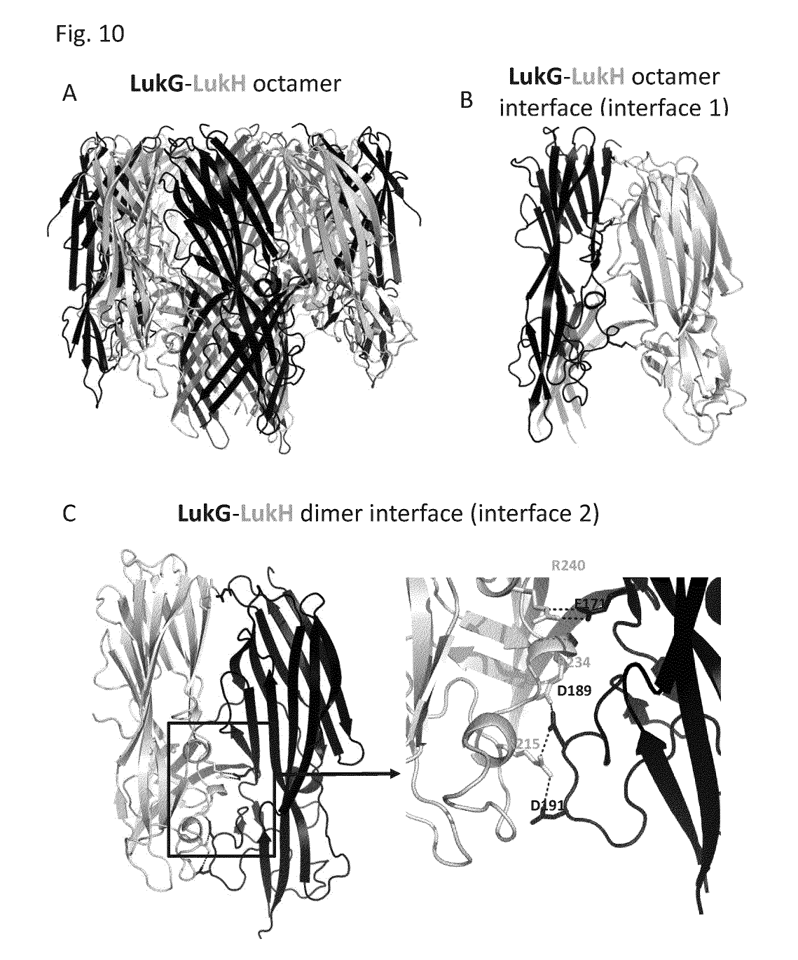

Six S. aureus toxins—LukH_TCH1516, LukH MRSA252, LukH MSHR1132, LukG_TCH1516, LukG_MRSA252 and LukG_MSHR1132—were produced recombinantly in E. coli (BL21, Rosetta or Tuner DE3). Toxin genes for the mature proteins (determined using the SignalP 4.1 Server; http://www.cbs.dtu.dk/services/SignalP/) were codon optimized for E. coli expression and generated by gene synthesis based on published genome sequences of Staphylococcus aureus strains USA300_TCH1516, MRSA252 and MSHR1132 (FIG. 1A, SeqIDs 1 to 12, FIG. 10). All toxins were expressed without tags in insoluble form, the proteins were refolded from inclusion bodies and purified; purification consisted of two steps on the size exclusion column for LukH and one step on cation exchange and one on anion exchange at pH 10.2-11.0 for LukG. The proteins were assayed for purity (by SDS-PAGE) and monomeric state (by size exclusion), as well as for functionality in in vitro assays, as described in Example 3 (FIG. 1B). All proteins were labeled with the amino reactive reagent Sulfo-NHS-LC biotin.

### Example 2

### Selection of LukG and LukH Binding Human Monoclonal Antibodies

Toxin binding antibodies were selected by yeast surface display libraries developed according to WO2009/036379A2, WO2012009568 and WO2010105256. Toxin molecules were expressed as recombinant E. coli produced proteins and labeled with biotin, as described in Example 1

A library of yeast cells engineered to express full length human IgG1 antibodies with an approx. 10⁹⁻¹⁰ diversity were incubated with biotin labeled toxins at different concentrations. Yeast cells expressing antibodies with the capacity of binding to the toxins were isolated by magnetic bead selection and fluorescence-activated cell sorting (FACS) employing streptavidin secondary reagents in several successive (up to five) selection rounds. Antibodies were then produced by the selected yeast clones and purified by Protein A affinity chromatography. Binding of individual, soluble mAbs to the different toxins was confirmed by interferometry measurements using a ForteBio Octet Red instrument [Pall Life Sciences]; the biotinylated antigen or the antibody was immobilized on the sensor and the association and dissociation of the antibody Fab fragment or of the antigen, respectively (typically 200 nM), in solution, were measured. The affinities (Kd values) were calculated based on the measured kinetic parameters (kon and koff).

### Example 3

### Analysis of Human mAbs for their LukGH Neutralizing Activity

The neutralizing activity of human mAbs—selected with LukH_TCH1516 and LukG_TCH1516, as described in example 2—against the LukGH toxin was assessed in a viability assay with human neutrophils. For this purpose neutrophils were isolated from fresh human whole blood, either obtained from the Red Cross (heparinized) or obtained by venipuncture from normal healthy volunteers in K-EDTA vacutainer tubes (BD, USA). To aggregate erythrocytes 1 part HetaSep solution (Stem Cell Technologies, France) was added to 5 parts of blood, mixed and incubated at 37° C. until the plasma/erythrocyte interphase was at approximately 50% of the total volume. The leukocyte enriched plasma layer was carefully layered on a 2-step Percoll gradient (73% and 63% Percoll Plus diluted in HBSS, GE Healthcare) and centrifuged at 680×g, RT, 30 min, no brakes. The first and second layers of the post-spin gradient (mainly serum and monocytes) were removed by aspiration. Neutrophils were harvested from the second opaque layer and washed twice in 50 ml HBSS (Gibco, USA)+10 mM Glucose. The number of viable cells was counted using trypan blue dye exclusion in a hemocytometer. The described isolation method usually yielded 1-5×10E8 neutrophils with a viability >95% out of 50 ml whole blood. For viability assays, cells were re-suspended in RPMI 1640 (PAA Laboratories, Austria) supplemented with 10% FCS, L-Glutamine and Pen/Strep (=neutrophil medium). Alternatively, the HL-60 (ATCC CCL-240™) human promyelocytic leukemia cell line was also used as source of neutrophil-like cells (Gallagher, Blood, 1979:713; Collins, PNAS, 1978:2458). The cells were cultured in neutrophil medium and differentiated with DMF (N,N-Dimethylformamide, 100 mM) for 5 days, as described by Romero-Steiner, Clin Diagn Lab Immunol, 1997:415. Differentiation was determined by disappearance of CD71 and appearance of CD11b staining using Brilliant Violet 421 conjugated anti-CD11b (clone ICRF44, BioLegend, USA) and PE-conjugated anti-CD71 monoclonal antibodies (clone OKT9, eBioscience, USA), according to standard methods described in the literature (e.g. Collado-Escobar, Biochem J, 1994:553; Trayner, Leuk Res, 1998:537; Watanabe, J Leuk Biol, 1993:40).

Monoclonal antibodies were serially diluted in neutrophil medium and mixed with toxins at a fixed concentration that decreased cell viability >95% [˜1 nM, 60 ng/ml]. Viability assay was started after a 30 minutes pre-incubation step to allow antibody binding to the toxin. 25,000 cells were added per well and the reaction was incubated for 4 hours at 37° C., in humidified atmosphere with 5% CO2. Viability of PMNs assessed using a commercial cell viability assay kit based on luminescent measurement of cellular ATP-levels (CellTiter-Glo® Luminescent Cell Viability Assay; Promega, USA) according to the manufacturer's instructions. % viability was calculated relative to mock-treated controls. % inhibition of toxin activity was calculated using the following formula: % inhibition=[(viability toxin only−inhibited activity)/(viability toxin only)]×100. The control mAb (generated against an irrelevant antigen: hen egg lysozyme) was included in all assays.

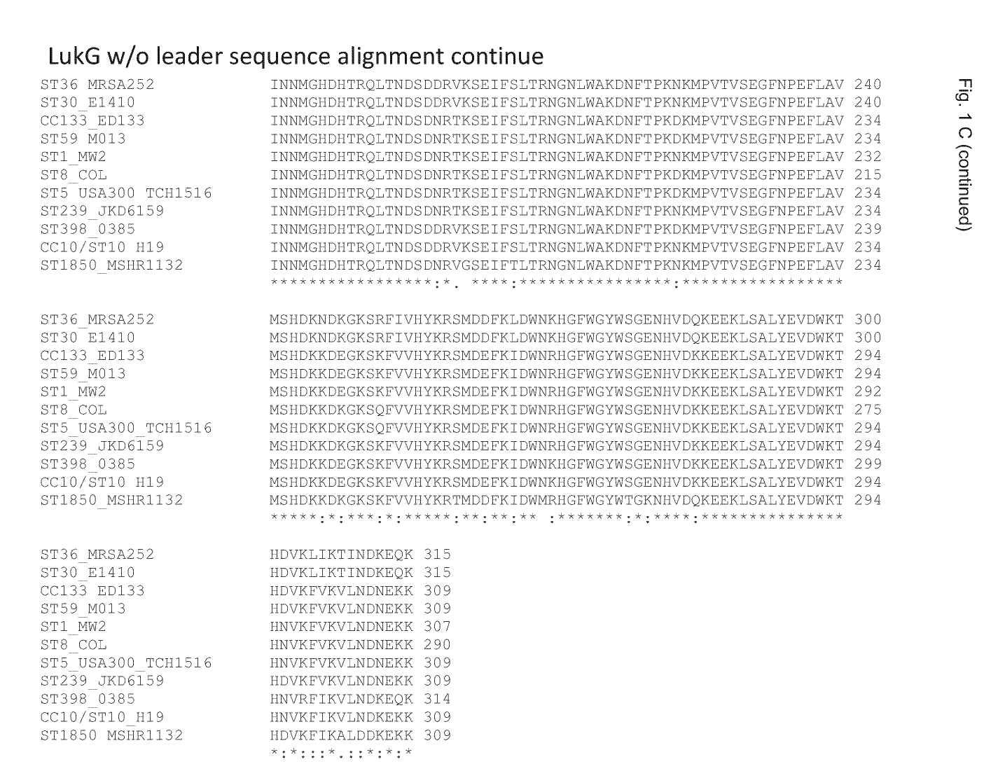

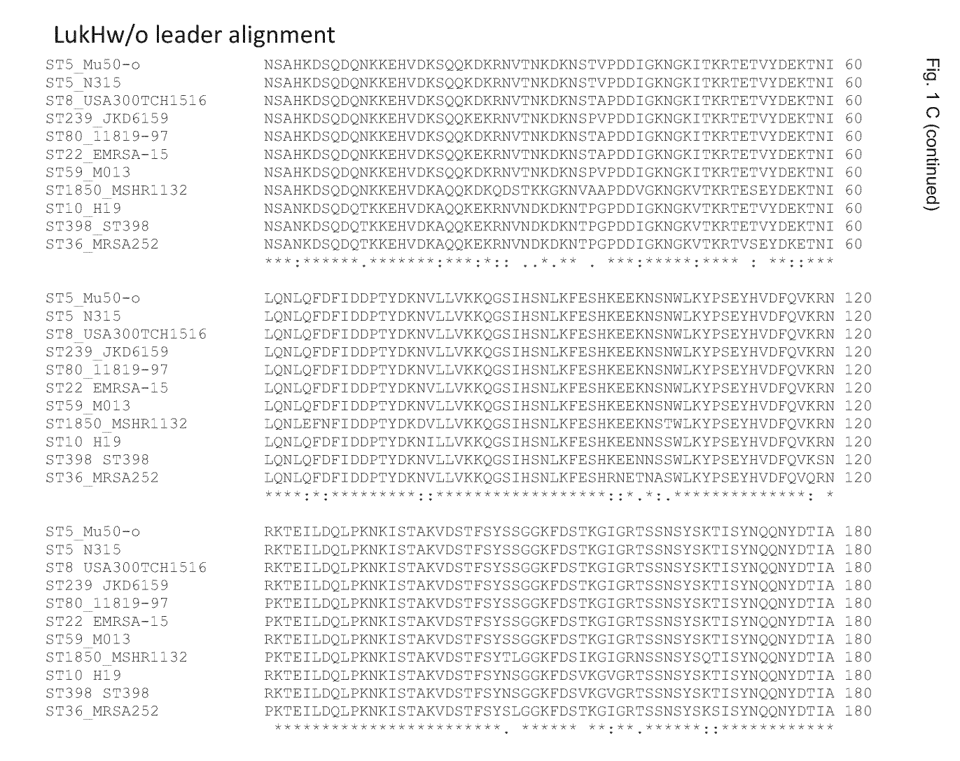

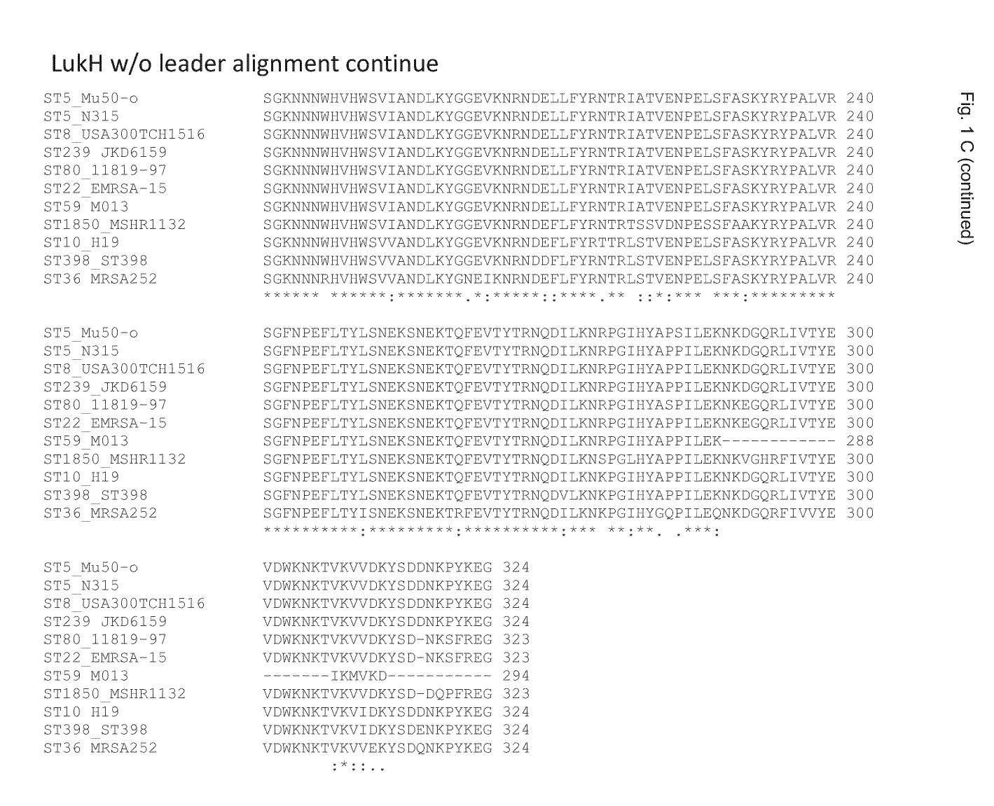

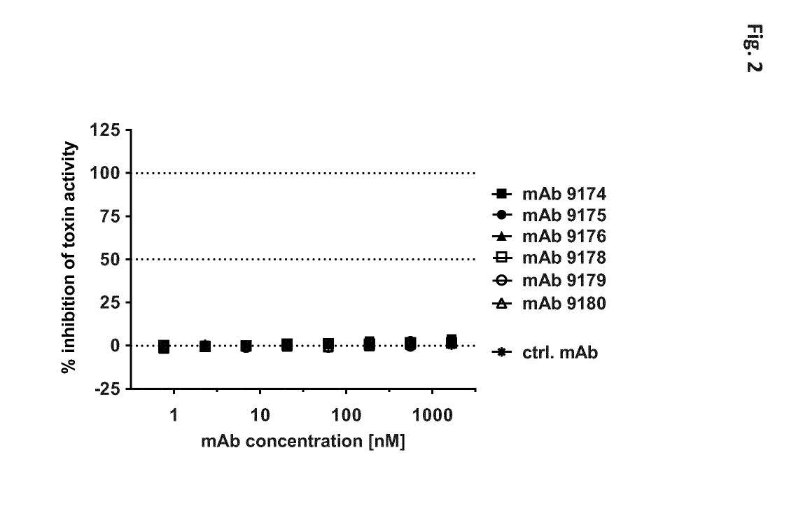

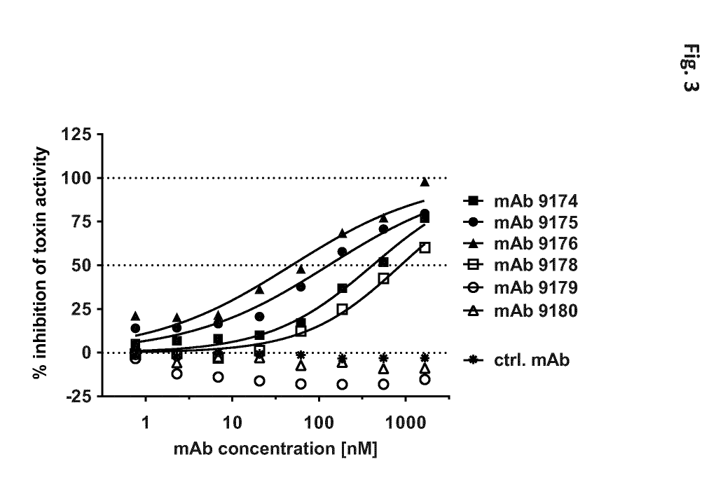

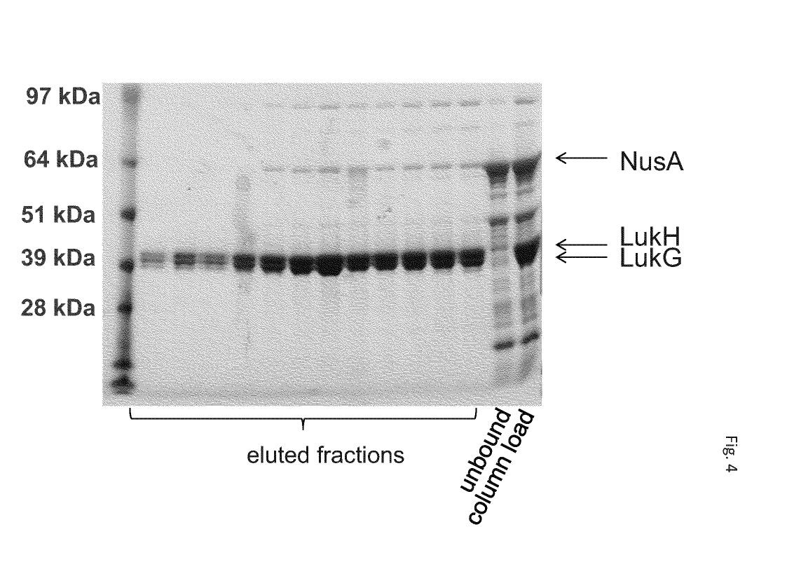

Using this method we could not identify potent neutralizing antibodies among those generated with the LukG or LukH toxin components (examples shown in FIG. 2). In an attempt to look for explanation, we hypothesized that the selected antibodies might not be able to bind to the cognate toxin component in the presence of the other component. In a series of binding studies with forteBio measurement, we established that LukG mAbs were inhibited to bind to LukG in the presence of LukH, and vica versa, the LukH mAbs were inhibited to bind to LukH in the presence of LukG. To confirm the suspicion that the mAbs were generated against protein domains that were in contact between LukH and LukG, the neutralization assay was performed with a modification. mAbs were pre-incubated with the cognate toxin component alone, before addition of the other toxin component. Indeed, a great improvement in toxin neutralization was seen with most of the LukG and LukH mAbs (examples shown in FIG. 3). These antibodies, however, were not able to neutralize the native toxin produced by S. aureus and present in bacterial culture supernatants. All these experiments suggested that the LukG and LukH formed a complex in solution before binding to target cells that has not been reported for the S. aureus bi-component leukocidins.

### Example 4

### Recombinant LukG and LukH Co-Purify from E. coli Lysates when Co-Expressed and Form Dimer in Solution

We co-expressed the LukH and LukG components in the same E. coli cell by co-transfection with two plasmids containing different antibiotic resistance markers, and each carrying either the lukH or the lukG gene. LukG was expressed as a fusion protein with NusA/His6 at the N-term to allow metal affinity purification of the complex, while LukH was expressed in un-tagged form. Induction of expression was done with isopropyl-β-D-thiogalactopyranoside (IPTG) for 20 h at 20° C. The two proteins were found in the soluble fraction and were co-purified by immobilized metal ion affinity chromatography (IMAC). The NusA/His6 tag was removed proteolytically (with enterokinase) giving the un-tagged, mature LukGH complex which was further purified by cation exchange chromatography.

Co-expression stabilized the individual proteins. While the individual components were always expressed in the insoluble fraction of E. coli (see example 1), the co-expressed LukGH was found in the soluble fraction.

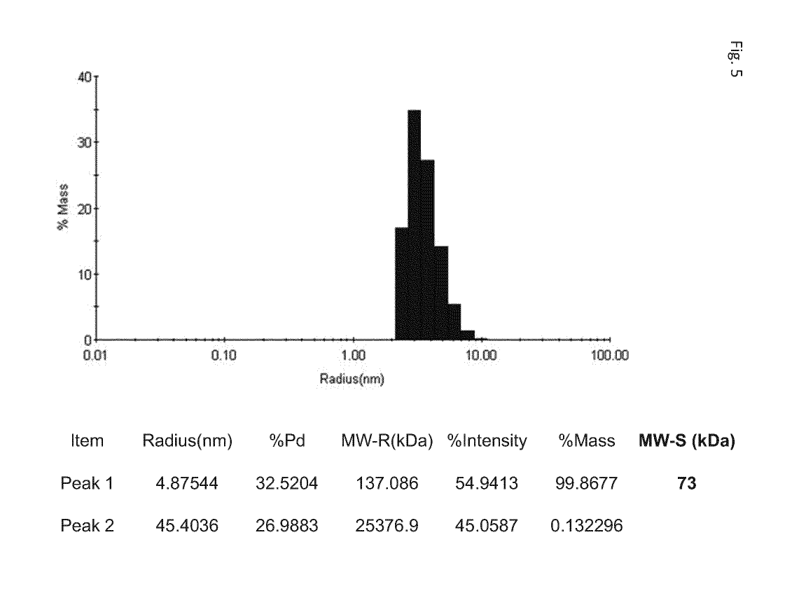

SDS-PAGE indicated that the stoichiometry of LukG: LukH in the complex is 1:1. To determine the size of the complex in solution we employed dynamic light scattering (DLS) measurements using a DynoPro NanoStar (Wyatt) instrument equipped with a static light scattering detector (FIG. 5). The molecular weight measured with the static light scattering detector (MW-S), is in excellent agreement with the calculated molecular weight of the heterodimer, 73 kDa.

### Example 5

### Highly Potent LukGH Neutralizing mAbs are Selected with the Recombinant LukGH Complex

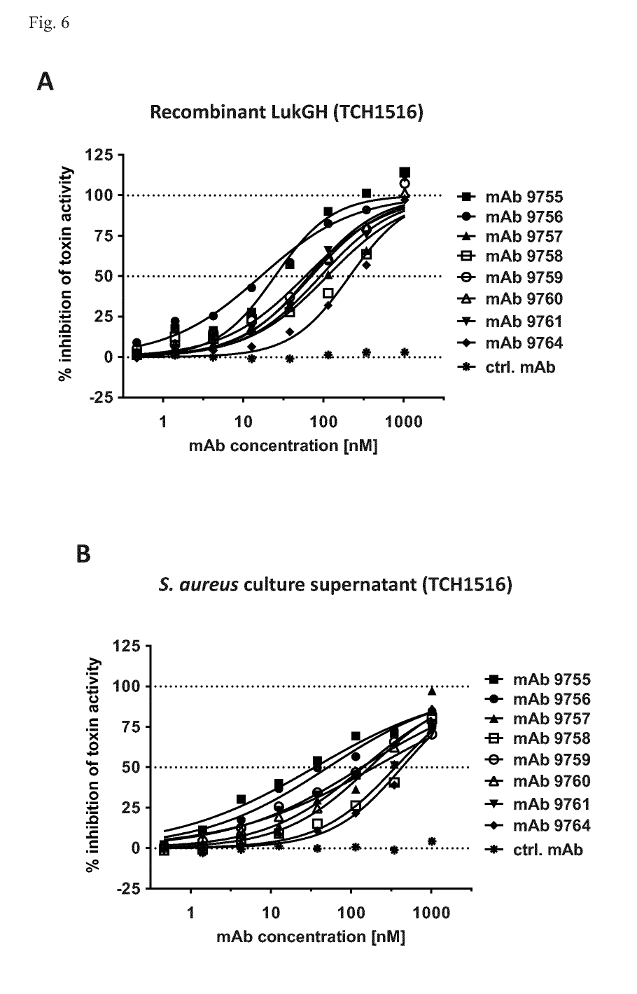

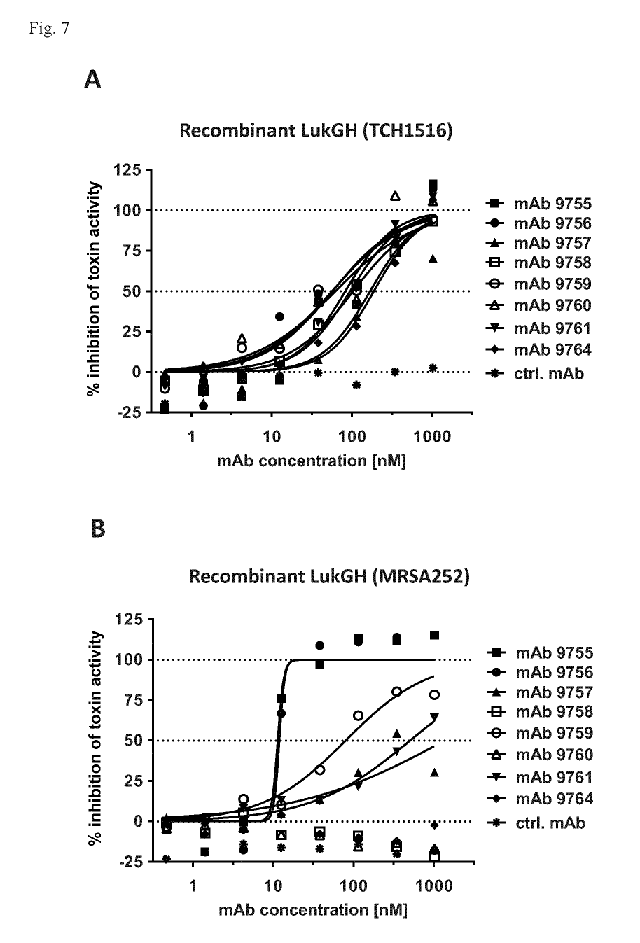

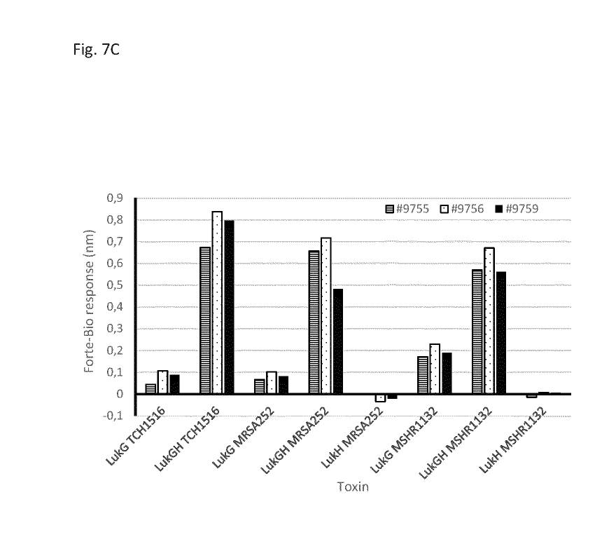

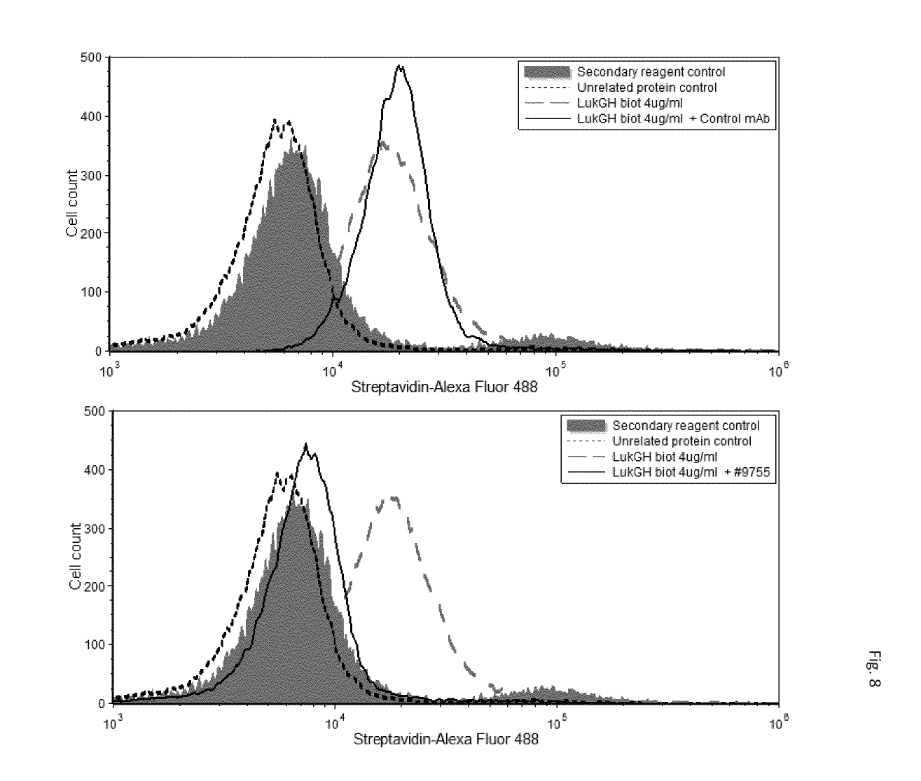

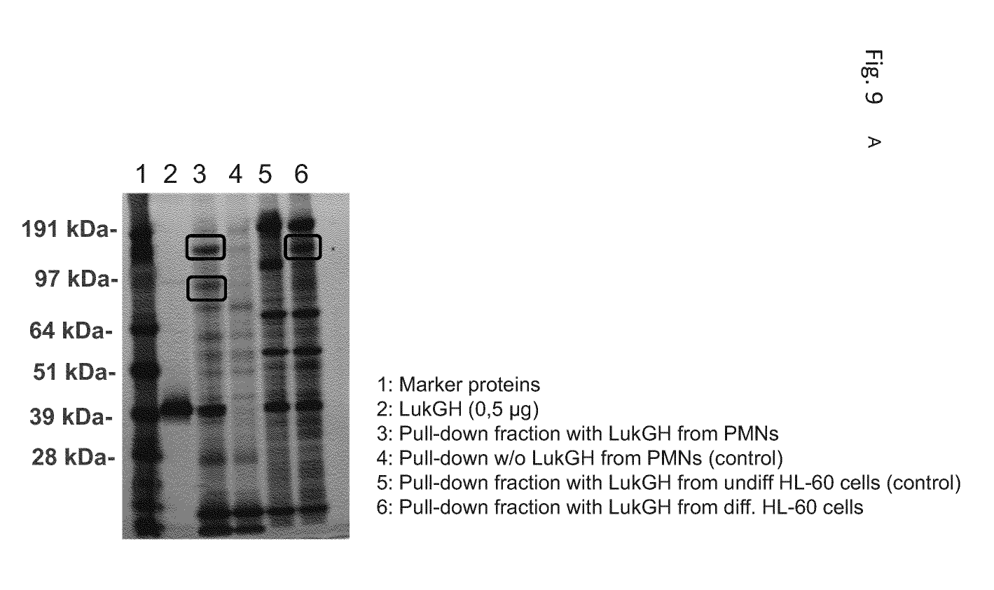

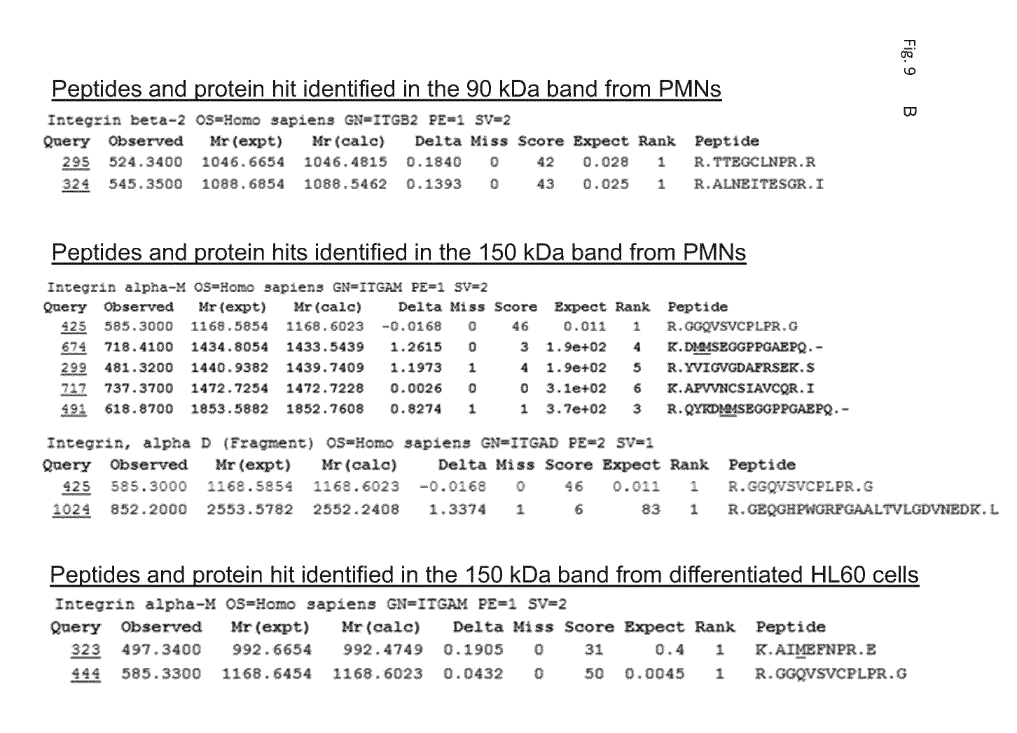

Antibody selection was performed by yeast surface display in the same manner described in example 2 using biotinylated LukGH complex as antigen. 84 human mAbs with unique CDR sequences were selected. Neutralization activity was measured as described in example 3 using either differentiated HL-60 cells or freshly isolated human PMNs and pre-incubation of antibodies was done with the highly potent LukGH complex or native LukGH produced by S. aureus cells (culture supernatant) cultured in RPMI-1640 medium supplemented with 1% casamino acids. In contrast to the results obtained with LukG or LukH mAbs, antibodies selected with the LukGH complex were proven to be potent, ¾ of them displaying half maximal inhibition concentration (IC50) below 300 nM. The most efficacious mAbs had IC50<30 nM (approximately 10 mAb:toxin ratio). Examples are shown in FIG. 6 either with recombinant LukGH complex (FIG. 6A) or with native LukGH FIG. 6B) using differentiated HL-60 cells. The same rank order in terms of potency was found when freshly isolated human PMNs were used as targets cells (FIG. 7A).

Since LukGH exists in different variants, it is an important aspect to select mAbs that are able to neutralize all LukGH variants expressed by different S. aureus strains. For that purpose, the most divergent sequence variant LukGH, expressed by the MRSA252 strain—that belongs to CC30 (clonal complex) and ST36 (sequence type)—was also tested in the toxin neutralization assays. This assay has a great differentiation power, as certain mAbs proved to be highly potent, while others did not display functionality (FIG. 7B). Potency was proven to correlate with binding to the LukGH complex variants based on the response values in ForteBio measurements when the antibodies were immobilized on anti-human capture sensors and the association of the toxins in solution (100 nM in PBS+3% BSA) was monitored. Binding of the mAbs to the LukGH complex variants is significantly better than to the LukG or LukH variants (FIG. 7C).

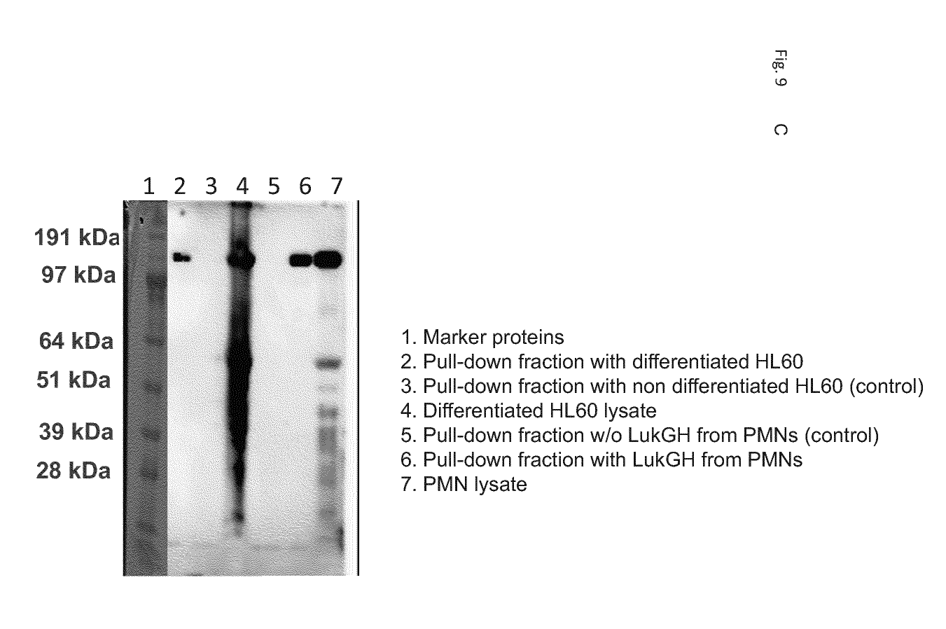

To uncover the mode of action of the LukGH neutralizing antibodies, binding of the biotinylated LukGH complex was monitored in the absence or presence of the LukGH mAbs using fluorescent Streptavidin (binding to biotin) in a flow cytometry based surface staining assay. The presence of antibodies correlated with lack of fluorescent surface signal suggesting that binding to the putative receptor was inhibited upon mAbs binding to the toxin (FIG. 8).

In conclusion, the discovery of complex formation by LukG and LukH in solution led to the identification of potent toxin neutralizing antibodies that protect human phagocytic cells from lysis. A subgroup of antibodies has a broad binding specificity and cross-neutralize the most different LukGH sequences. The neutralizing LukGH mAbs inhibit binding of the toxin to phagocytic cells and thus prevent lysis. These characteristics of mAbs render them suitable for broad clinical applications.

### Example 6

### Identification of the CD11b/CD18 Complex as the Human Receptor for LukGH

We determined with flow cytometry based surface staining that the LukGH complex bound to human PMNs and differentiated HL-60 cells, but not to undifferentiated HL-60 cells. It correlated to toxin sensitivity as the latter cell type was completely resistant to LukGH (no cell lysis detected). These data suggested that the LukGH receptor was expressed by PMNs and differentiated HL-60 cells. To identify this receptor a pull down experiment was performed using biotin labeled LukGH (2 μg) and 10⁸ cells. The biotinylated LukGH and its binding partner(s) were collected on Streptavidin agarose resin (binding biotin) and analyzed by SDS-PAGE. Two unique protein bands with molecular masses 150- and 90-kDa appeared in the PMN and differentiated HL-60 pull-down samples, while missing from the undifferentiated HL-60 fraction and also from PMN samples purified without adding LukGH (FIG. 9A). These bands were cut out from the gel and subjected to mass spectrometric analysis (peptide mass fingerprinting). The gel bands were digested (trypsin), the peptides obtained measured by nano-LC-MS/MS and the MS/MS spectra were interrogated against the Mascot (http://www.matrixscience.com) database. Based on the mass of the tryptic peptides, the two proteins were identified as CD11 b (150-kDa) and CD18 (90-kDa) (FIG. 9B). CD11 b and CD18 are known to form a complex on the surface of PMNs and differentiated HL-60 cells, also called the complement receptor 3 (CR3), or leukocyte integrin Mac-1 (FEMS Immunol. Med. Microbiol. 2002, 34, 255). Presence of CD11 b in the PMN and differentiated HL-60 pulled down material was confirmed in an western blot with an anti-CD11b specific mAb (Abcam ab52478), (FIG. 9C).). It is an intriguing finding and fully explains the cell type specificity of LukGH, reported to be toxic to human PMNs, monocytes and dendritic cells, all known to express CD11b/CD18. It is also widely established that undifferentiated HL-60 cells lack surface expression of this complex. Actually, positive CD11b/CD18 staining is routinely used as a marker of efficient differentiation of HL-60 cell in vitro. Peptide mass analysis has also identified, with a lower score, CD11d in the 150 kDa band isolated from PMNs (FIG. 9B). CD11d also forms a complex with CD18 to give integrin αDβ2, which was shown to be up-regulated on inflammatory macrophages and modulate macrophage adhesiveness and their migration (Exp. Cell. Res. 2008, 314, 2569). αDβ2 is 58% identical to CR3 at amino acid level and is a potential alternative receptor of LukGH. These data also explains the lack or very low toxin activity towards mouse and rabbit cells (reported in the literature, Malachowa et al, 2012 and confirmed by us), since the rodent CD11b/CD18 shares only ˜75% amino acid identity with their human counterpart. Likewise, human CD11d is only ˜70% identical to the mouse variant.

### Example 7

### Crystal Structure of the LukGH Octamer Identifies the Residues Important for Dimer Formation

The structure of LukGH USA300 solved at 2.8 Å resolution revealed an octameric pore arrangement, with two octamers in the asymmetric unit, each octamer being composed of four alternating LukG and LukH subunits (FIG. 10A), similar to that obtained with HIgAB (PNAS 108, 17314, 2011). The toxin protomers are involved in two types of interfaces in the octamer: the interface between chains A and B (interface 1) and that between chains B and C (interface 2). The buried surface areas are 2188 and 2461 Å², for interfaces 1 and 2, respectively. In interface 1 the main interactions occur between the cap domains (FIG. 10B), whereas in interface 2 there are interactions both between the cap domains and the rim domains of the two monomers (FIG. 10C). Analysis of the contact residues between the two interfaces in the LukGH octamer has shown that interface 1 is stabilized by 34 hydrogen bonds and a series of electrostatic interactions involving residues Asp39, Asp75 and Asp197 from LukH and Lys56, Lys58, Arg23 and Lys 218 from LukG. In interface 2, there are altogether 56 hydrogen bonds, with electrostatic interactions involving residues Arg49 and Arg240 from LukH and Asp49 and Glu171 from LukG between the cap domains and residues Arg215 and Arg234 from LukH and Asp189 and Asp191 from LukG between the rim domains. When comparing to the salt bridges observed in the HIgAB octamer (pdb code 3B07), those found between the cap domains are mostly conserved between the LukGH and HIgAB octamers (and also between other S and F components), while those between the rim domains (in interface 2) are only found in LukGH and the residues involved are fully conserved between the LukGH variants (FIG. 1C). Based on the size of the buried surface areas in the two interfaces and on the lack of conservation of the salt bridge residues in interface 2 with the other F and S components (that are not known to form dimers in solution), the most likely interface for the LukGH dimer is interface 2 (FIG. 10C). To confirm this hypothesis we have generated interface mutants, by changing to Ala the residues involved in electrostatic interactions as follows. Asp75 and Asp197 in LukH were mutated to Ala to give LukH1 (interface 1 LukH mutant) and Arg23 and Lys218 in LukG were mutated to Ala to give LukG1 (interface 1 LukG mutant). To make the 2^{nd} interface mutants, LukH2 and LukG2, Arg215, Arg234 and Arg240 in LukH and Asp189, Asp191 and Glu171 in LukG, respectively, were mutated to Ala.

The LukGH variants were co-transformed and co-expression of the complexes was induced at 20° C. as for the WT complex. All complexes were co-expressed at similar levels, but the amount of soluble complex varied among different variants; the interface 1 mutants: LukG1H, LukGH1 and LukG1H1 showed similar solubility as the WT complex, while the interface 2 mutants: LukG2H, LukGH2 and LukG2H2 were mainly insoluble. Therefore the interface 1 mutant complexes were purified using a similar procedure as for the WT LukGH, and similar yields were obtained. The purified LukGH variants were checked for folding by circular dichroism (CD), and their CD spectra were essentially the same as for the WT complex, indicating that the mutations had no effect on the secondary structure of the complex.

The individual components: LukG1, LukG2 and LukH1 and LukH2 were also expressed and purified from inclusion bodies as for the WT LukG and LukH. Similar yields were obtained for LukG1, LukG2 and LukH2 as for the WT counterparts: the CD spectrum of LukH2 compares well with that of WT LukH, and the same is true for the LukG variants, although in the pH 10.0 buffer where the proteins were formulated and the spectra acquired, all LukG proteins show mainly random coil structures. The LukH1 yields were significantly lower than for the WT protein, supported by the fact that LukH1 is partly unfolded, and was therefore not included in further experiments.

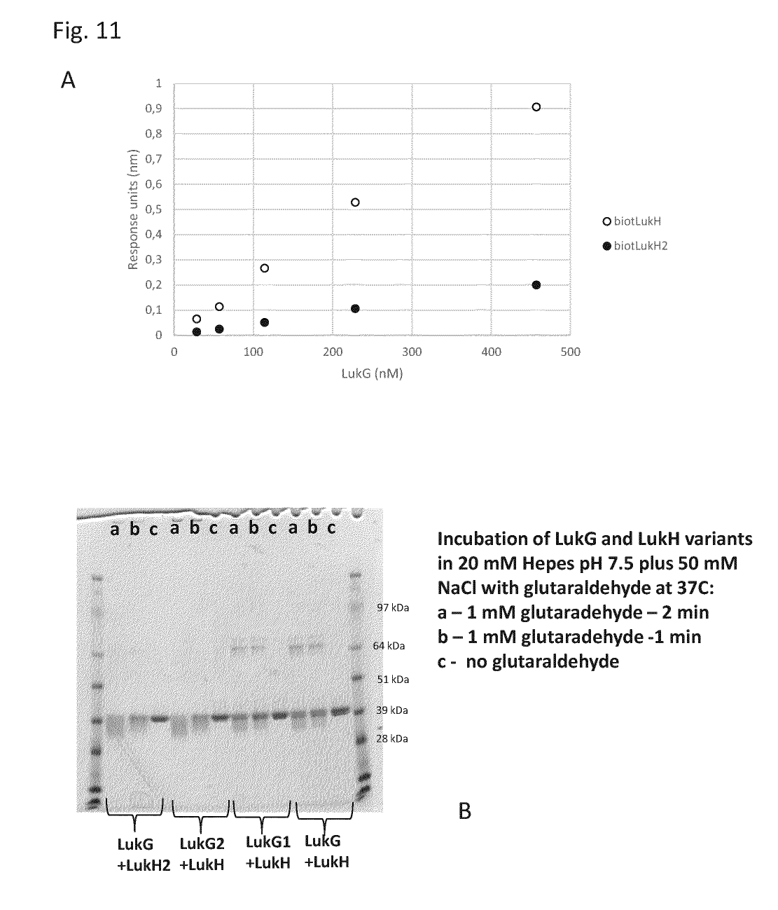

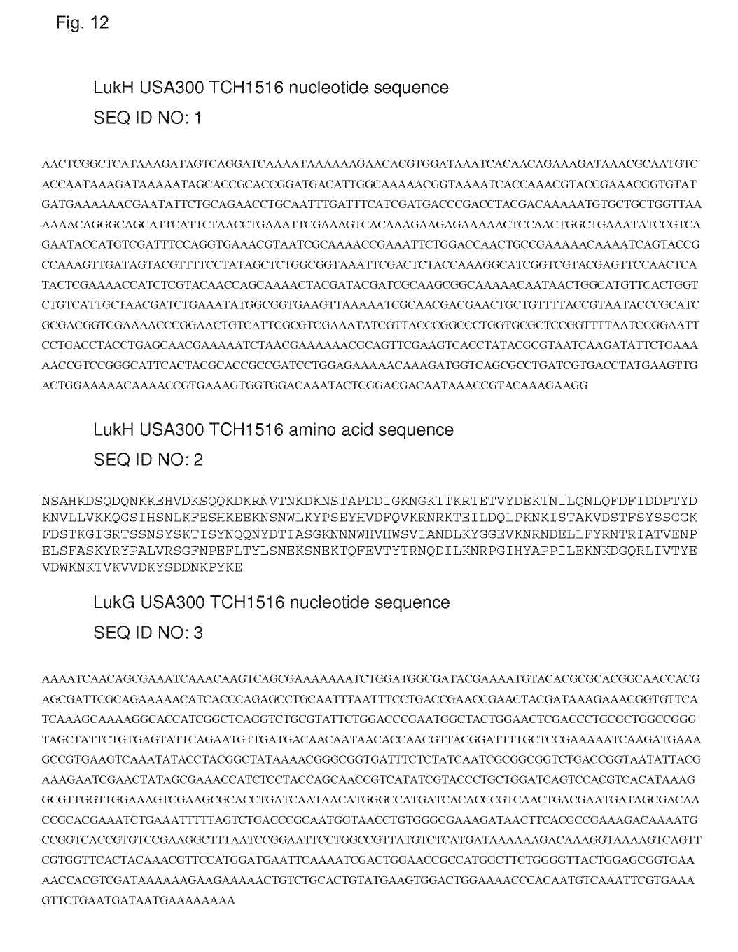

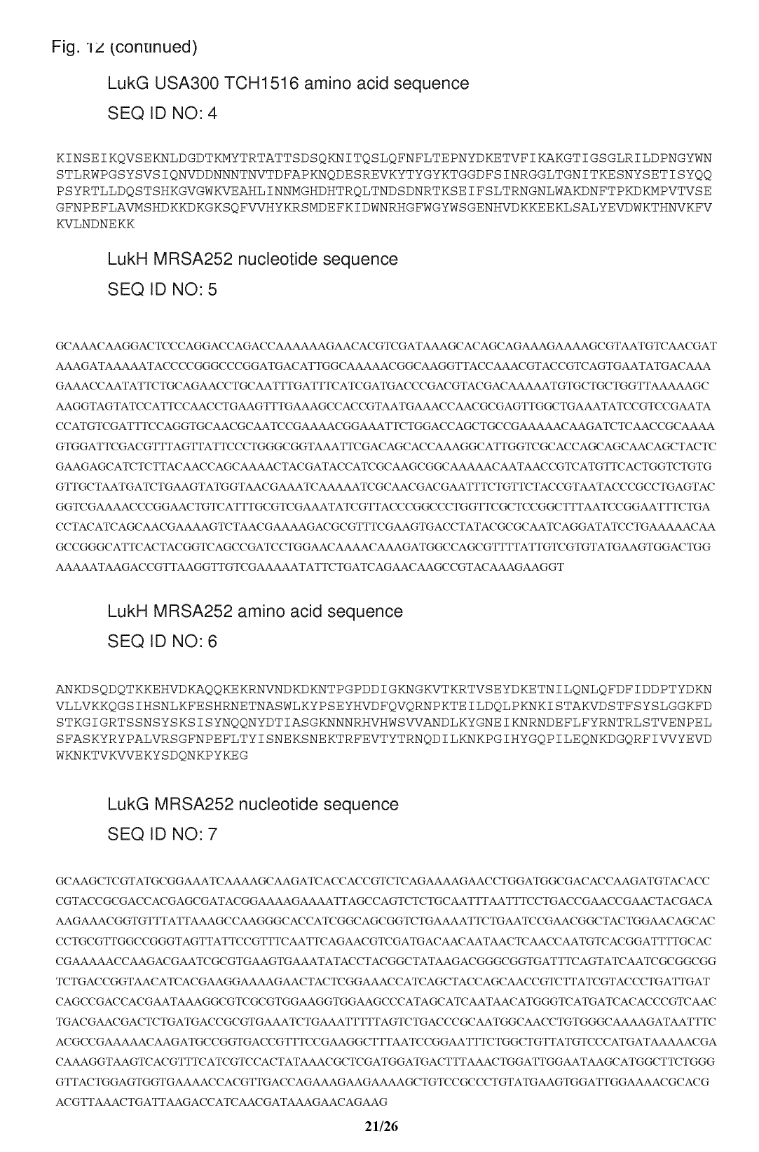

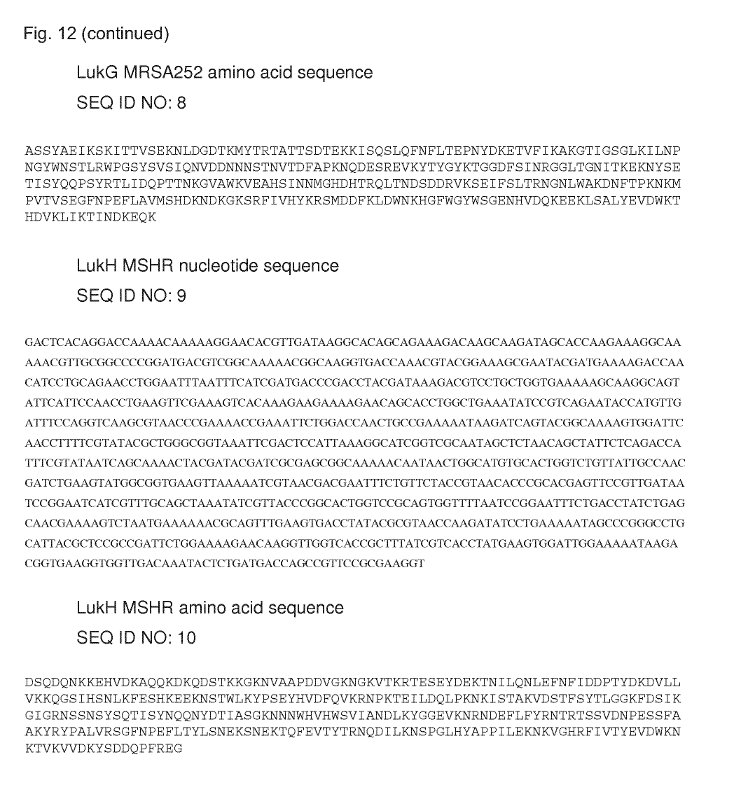

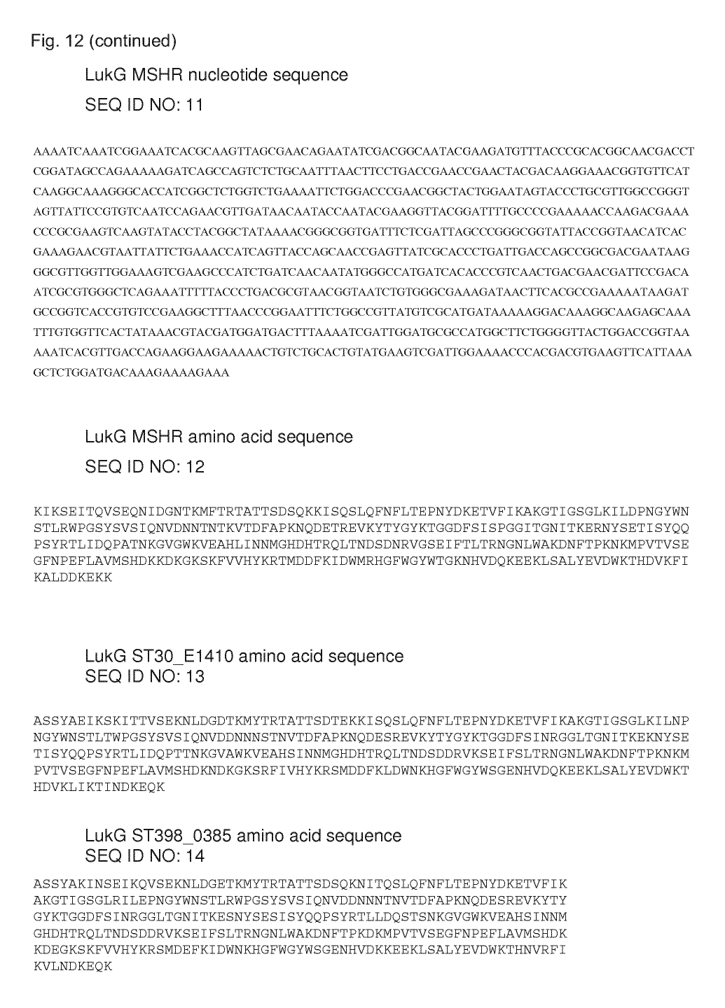

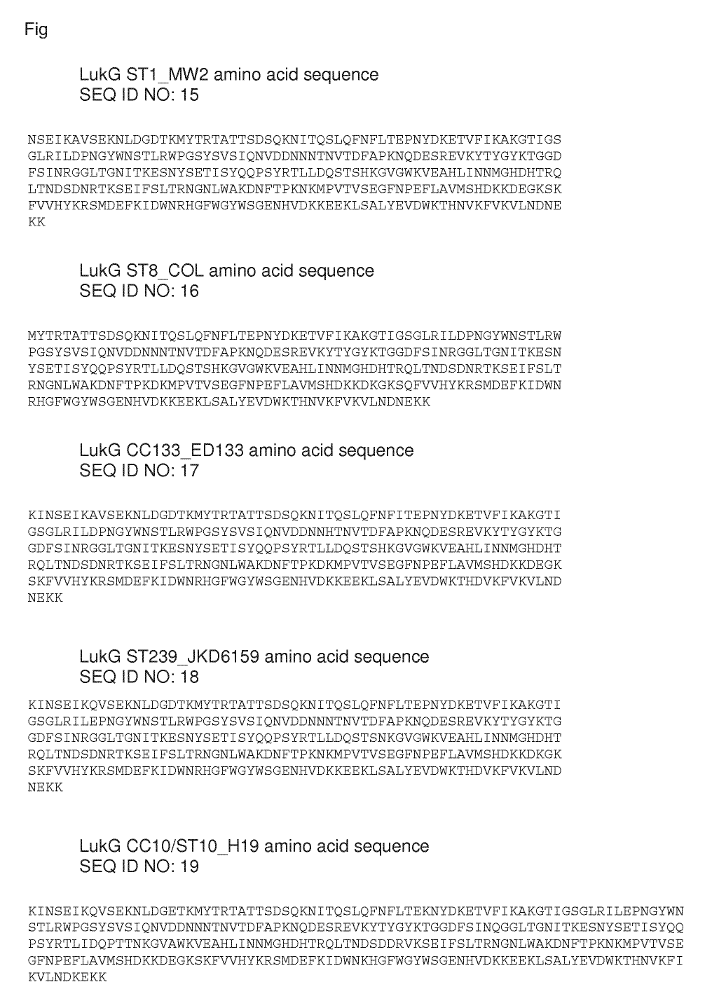

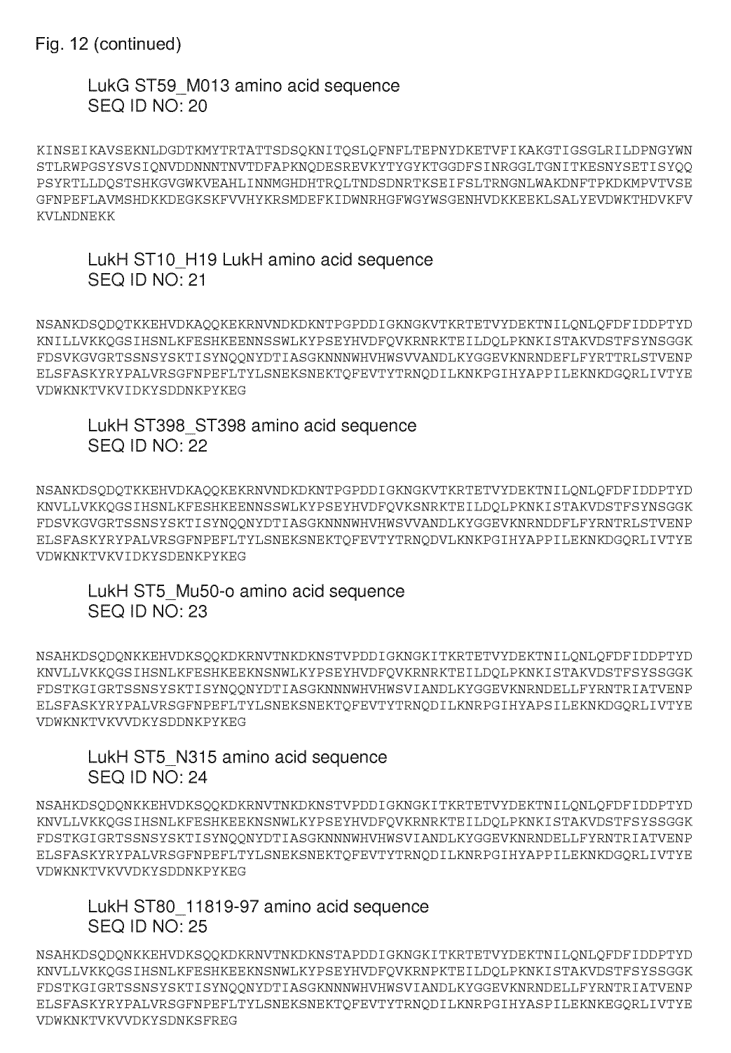

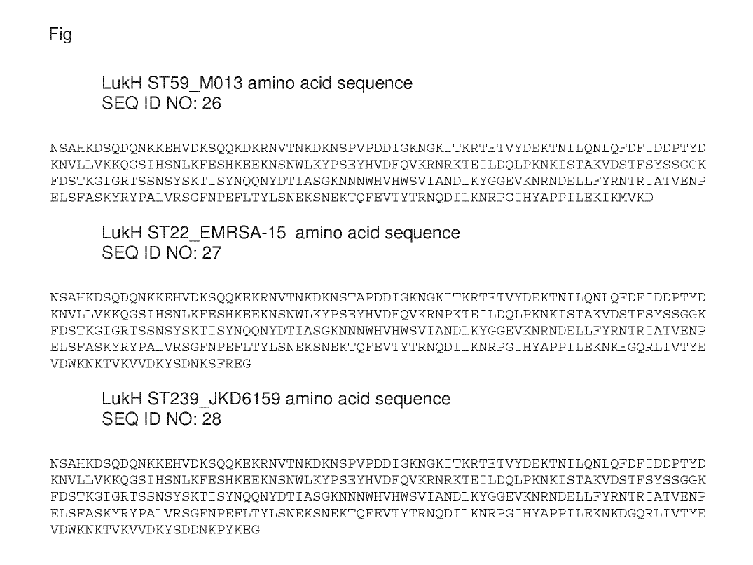

The formation of the LukGH complex from the individual components was monitored in Forte-Bio by immobilizing biotinylated LukH or LukH2 on streptavidin sensors and measuring the response values for the association of LukG to the preloaded sensors. There was significantly higher binding with LukH than with LukH2 (FIG. 11A) in agreement with the mutations in LukH2 affecting the dimer interface. Cross-linking of mixtures of LukG and LukH variants (35 μg/ml each) with glutaraldehyde (FIG. 11B) confirms that interface 2 mutants are unable to form the LukGH dimer, but the interface 1 LukG mutant is able to form the dimer at levels similar to those observed for the WT components.

### REFERENCES

Malachowa N, Kobayashi S D, Braughton K R, Whitney A R, Parnell M J, Gardner D J, Deleo F R. Staphylococcus aureus leukotoxin GH promotes inflammation. J Infect Dis. 2012 October; 206(8):1185-93. Epub 2012 Aug. 7.
Ventura C L, Malachowa N, Hammer C H, Nardone G A, Robinson M A, Kobayashi S D, DeLeo F R. Identification of a novel Staphylococcus aureus two-component leukotoxin using cell surface proteomics. PLoS One. 2010 Jul. 16; 5(7):e11634.
Dumont A L, Yoong P, Surewaard B G, Benson M A, Nijland R, van Strijp J A, Torres V J Staphylococcus aureus Elaborates Leukocidin A B To Mediate Escape from within Human Neutrophils. Infect Immun. 2013 May; 81(5):1830-41. doi: 10.1128/IAI.00095-13. Epub 2013 Mar. 18.
Dumont A L, Nygaard T K, Watkins R L, Smith A, Kozhaya L, Kreiswirth B N, Shopsin B, Unutmaz D, Voyich J M, Torres V J. Characterization of a new cytotoxin that contributes to Staphylococcus aureus pathogenesis. Mol Microbiol. 2011 February; 79(3):814-25. Epub 2010 Dec. 13.
Agramonte-Hevia A., FEMS Immunol. Med. Microbial. 2002, 34, 255
Yakubenko, V. P., Exp. Cell. Res. 2008, 314, 2569

1. An isolated Staphylococcus aureus leukocidin antigen comprising a LukGH complex.

2. The antigen of claim 1, wherein the LukGH complex comprises the LukG and LukH components as a dimer or oligomer.

3. The antigen of claim 1, wherein the LukGH complex is composed of recombinant proteins and/or proteins derived from S. aureus strains.

4. The antigen of claim 3, wherein the LukG and LukH components are co-expressed by a recombinant host cell, purified from native sources and/or co-refolded.

5. The antigen of claim 1, which is provided as a protein complex in the soluble form.

6. The antigen claim 1, which is capable of binding to the human CD11b/CD18 receptor.

7. An antibody specifically binding to the LukGH complex.

8. The antibody of claim 7, which is neutralizing the LukGH complex.

9. The antibody of claim 7, which is binding to the LukGH complex derived from a USA300 clone, preferably from the TCH1516 strain, and at least one of the LukGH complex variants.

10. The antibody of claim 7, wherein the LukGH complex variants have at least one point mutation in the amino acid sequences of any of the LukG or LukH components, as compared to the LukGH complex derived from the USA300 clone.

11. The antibody of claim 7, wherein the LukGH complex variants comprise a LukG component comprising an amino acid sequence selected from the group consisting of SEQ ID 8, 12, 13, 14, 15, 16, 17, 18, 19 and 20, and/or a LukH component comprising an amino acid sequence selected from the group consisting of SEQ ID 6, 10, 21, 22, 23, 24, 25, 26, 27 and 28.

12. The antibody of claim 7, wherein the LukGH complex derived from a USA300 clone comprises the LukG component comprising the amino acid sequence of SEQ ID 4 and/or the LukH component comprising the amino acid sequence of SEQ ID 2.

13. The antibody claim 7, which is cross-neutralizing the LukGH complex and the LukGH complex variants.

14. The human CD11b/CD18 complex for use in a method of determining the binding or toxicity of the Staphylococcus aureus LukGH bi-component cytolysin.

15. The CD11b/CD18 complex for use according to claim 14, which is used in its native form, or isolated or immobilized forms.
